SEQUENCE LISTING (6 sequences)

SEQ ID NO: 1 - DNA, 20 bp, Artificial
  Synthetic Construct oligonucleotide useful as primer
aggtttgaagctgcttcttc

SEQ ID NO: 2 - DNA, 20 bp, Artificial
  Synthetic Construct oligonucleotide useful as primer
cgctgcatgtgccattaaga

SEQ ID NO: 3 - DNA, 20 bp, Artificial
  Synthetic Construct oligonucleotide useful as primer
tctacaacgttatcgtcaca

SEQ ID NO: 4 - DNA, 20 bp, Artificial
  Synthetic Construct oligonucleotide useful as primer
tgcagtgctctgagccctag

SEQ ID NO: 5 - DNA, 20 bp, Artificial
  Synthetic Construct oligonucleotide useful as primer
ctaggtgtaaggagaagatg

SEQ ID NO: 6 - DNA, 20 bp, Artificial
  Synthetic Construct oligonucleotide useful as primer
gatttacgccgatgaatatg

(19) United States
(12) Patent Application Publication
(10) Pub. No.: US 2008/0280294 A1
(43) Pub. Date: Nov. 13, 2008

(54) Inherited Mitochondrial Dna Mutations in Cancer

(75) Inventors: John Petros, Norcross, GA (US); Amanda Baumann, Durham, NC (US); Douglas C. Wallace, Irvine, CA (US); Carrie Sun, Tucker, GA (US); Muta Issa, Atlanta, GA (US); Fray F. Marshall, Atlanta, GA (US)

Correspondence Address: GREENLEE WINNER AND SULLIVAN P C, 4875 PEARL EAST CIRCLE, SUITE 200, BOULDER, CO, 80301, US

(73) Assignee: EMORY UNIVERSITY, ATLANTA, GA (US)

(21) Appl. No.: 11/813,660

(22) PCT Filed: Dec. 27, 2005
(86) PCT No.: PCT/US2005/046908

§ 371 Date: Jun. 2, 2008

Related U.S. Application Data
(60) Provisional application No. 60/642,743, filed on Jan. 10, 2005.
(60) Provisional application No. 60/666,752, filed on Mar. 30, 2005.

(51) Int. Cl. C12Q 1/68; C07H 21/00

(52) U.S. Cl. 435/6; 536/24.5

(57) ABSTRACT

A method is provided for identifying a subject likely to have, or at risk of developing a disease condition correlated with increased reactive oxygen species (ROS), including cancer, by identifying in the subject a missense mutation in a nucleic acid of Complex III, IV and/or V of the OXPHOS system. This invention also provides a method of identifying a likelihood of having a heritable predisposition to cancer by detecting a homoplasmic missense mutation in non-tumor tissue of an OXPHOS system gene. This invention also provides a method for detecting likelihood of having cancer, predisposition to cancer, and likelihood of passing a predisposition to cancer to progeny involving identifying in non-tumor tissue of the subject a missense mutation in a complex III, IV and/or V gene of the mitochondrial OXPHOS system. The mutation may be a nuclear or mitochondrial mutation. The invention has been exemplified with respect to prostate cancer. When the mutation is homoplasmic in non-tumor tissue this is an indication it is an inherited and inheritable trait, and that the subject is likely to pass on the mutation to her progeny in the case of mutations in mitochondrial DNA or his or her progeny in the case of mutations in nuclear DNA. Both homoplasmic and heteroplasmic mutations in non-tumor tissue can indicate the presence of cancer.

### CROSS-REFERENCE TO RELATED APPLICATIONS

This application claims priority to U.S. Provisional Application Ser. No. 60/666,752 filed Mar. 30, 2005 and Ser. No. 60/642,743 filed Jan. 10, 2005, both of which are incorporated herein or by reference to the extent not inconsistent herewith.

### STATEMENT REGARDING FEDERALLY SPONSORED RESEARCH OR DEVELOPMENT

This invention was made with government support under NIH grant CA96994, CA98912, NS21328 and AG13154, and Department of Defense grants DAMNS17-00-1-0080. The government has certain rights in the invention.

### BACKGROUND OF THE INVENTION

In 2005, 232,090 new prostate cancer and 36,160 new renal cancer cases were predicted with deaths of 30,350 men from prostate cancer, and 12,660 deaths from renal cancer¹. While other cancers (e.g. leukemia, lymphoma, testicular carcinoma) are characterized primarily by excessive cellular proliferation, both prostate and renal cancers are characterized primarily by resistance to apoptosis^{2,3}. The mitochondrion is a key player in the process of apoptosis.

There is increasing evidence that mitochondrial gene mutations are associated with various cancers, but their pathophysiological significance remains unclear. The first clear demonstration that mitochondrial DNA (mtDNA) mutations in cancer could have functional significance came with the report of a middle aged woman with a renal adenocarcinoma that was heteroplasmic (mixture of mutant and normal mtDNAs) for a 294 nucleotide pair (np) deletion in the mtDNA Oxidative Phosphorylation (OXPHOS) gene ND1⁴. Subsequently, a variety of mtDNA coding region and control region (CR) mutations have been reported in cancers such as prostate cancer⁵⁻⁸, and a variety of other solid tumors⁹.

Gene amplification was found in human gliomas¹⁰ and point mutations were found in colorectal and gastric carcinomas¹¹. Homoplasmic point mutations were found in 7 of 10 colorectal tumors in one report¹² and frameshift mutations in another¹³. Loss of D-loop heteroplasmy occurs as a nearly universal feature of astrocytic brain tumors¹⁴. Because of the high copy of the mitochondrial genome, mtDNA mutations may be detected in the urine of patients with bladder cancer or the bronchial fluid of patients with lung cancer¹⁵. Because of reports highlighting somatic mutations and the severity of phenotype in “classic” inherited mitochondrial diseases, the prevailing paradigm for the relationship between mtDNA mutations and cancer has been restricted to somatically acquired mutations, especially cancer-specific mutations of this genome. There has been little consideration given to the possibility that missense mutations in peptide coding regions of the mtDNA may serve as an inheritable predisposition to the development of cancer.

Because the relative activities of glycolysis and oxidative phosphorylation (OXPHOS) are frequently altered in cancer, the mitochondrion has been implicated in the biology of cancer for decades¹⁶. Current understanding centers around the proven role the mitochondrion plays in the first committed step in apoptosis, the release of cytochrome c¹⁷. In addition, reactive oxygen species (ROS) are a natural by-product of mitochondrial OXPHOS, and ROS have mutagentic as well as mitogenic effects relevant to malignant transformation¹⁸.

Mutations in nuclear DNA (nDNA)-encoded mitochondrial genes have also been linked to cancer. Mutations in the nDNA-encoded succinate dehydrogenase (SDH) B, C, and D subunits of OXPHOS complex II have been linked to paragangliomas¹⁹⁻²². However, mutations in SDH A, the succinate-binding subunit, have been linked to Leigh Syndrome²³, not paraganglioma, demonstrating that transformation due to complex II mutants is not simply the result of energy deficiency.

Increased reactive oxygen species (ROS) production has been proposed to be an important factor in tumor formation in association with inactivation of p16ink4a and p53²⁴.

Reactive oxygen species include superoxide anion (O₂⁻), hydrogen peroxide (H₂O₂), and hydroxyl radical (OH⁻). Under normal conditions, the primary intracellular source of ROS is the mitochondria where O₂⁻ is generated as a by-product of electron transport and oxidative phosphorylation. A second major source is the enzymatic generation of O₂⁻ by oxidases, including NADPH oxidase, lipoxygenase, cyclooxygenase, xanthine oxidases, and others including non-phagocytic oxidases (NOX). In addition, growth factors use ROS as a second messenger. Superoxide is converted to H₂O₂ either spontaneously or by superoxide dismutase (SOD). Hydrogen peroxide is more stable than superoxide and can have a long half-life in biologic systems. In addition, H₂O₂ can freely diffuse across membranes while O₂⁻ cannot. Hydrogen peroxide can be converted to the highly reactive hydroxyl radical, a process that is enhanced in the presence of iron and other metal ions. Hydrogen peroxide can also be “detoxified” to water via the action of catalase or the glutathione peroxidase system.

The traditional view by cancer biologists of oxygen radicals and their highly reactive organic products is that these molecules contribute to malignancy by physical interaction with and disruption of large biomolecules, including DNA, RNA, proteins and lipids. In this way they can be highly mutagenic (causing DNA mutations) and can induce severe cellular damage, as is seen in phagocytes during the oxidative burst that is responsible for bacterial killing. While this is certainly the case, recent evidence suggests that there is a more subtle effect of at least some ROS as important second messengers involved in signal transduction, regulation of gene expression and cell-cell signaling. While it is known that ROS can induce mutations via DNA base alteration, it is also now clear that enzymatically generated ROS (specifically H₂O₂) is capable of mediating mitogenic effects as well²⁵.

Under normal conditions the mitochondria produce a small amount of reactive oxygen species (ROS). These are generated as an inevitable by-product of oxidative phosphorylation. The initial product is superoxide anion that is created when electrons exit the electron transport chain (located in the inner mitochondrial membrane) and are donated to molecular oxygen. This low level of superoxide production is detoxified primarily by mitochondrial superoxide dismutase (SOD2 MnSOD), which converts superoxide to hydrogen peroxide. Hydrogen peroxide is much more stable than superoxide and additionally has the ability to cross biologic membranes, which superoxide anion does not (or does so only at very low levels). The hydrogen peroxide produced in the mitochondria can enter the cytoplasm, altering multiple cell signaling pathways and may also enter the nucleus altering activity of transcription factors. Any perturbation that interferes with the efficient flow of electrons (such as mitochondrial DNA mutation) results in greater proportions of electrons being inappropriately donated to molecular oxygen, thereby increasing the rate of superoxide generation²⁶

As stated above, ROS are generated as a toxic by-product of mitochondrial (oxidative phosphorylation (OXPHOS). OXPHOS is composed of the electron transport chain (ETC), encompassing complexes I, II, III and IV, and the H⁺-transporting ATP synthase, complex V. Reducing equivalents (electrons) from dietary calories are passed down the ETC where they ultimately reduce O₂ (four electrons) to generate H₂O. The energy that is released is used to pump protons out across the mitochondrial inner membrane through complexes II, III, and IV to create an electrochemical gradient (ΔP=Δψ+Δμ^{H+}). AP is then used as a source of potential energy by complex V to condense ADP and phosphate (Pi) to generate ATP, and ATP is exchanged across the mitochondrial inner membrane for spent cytosolic ADP by the adenine nucleotide translocators (ANT)²⁷.

When calories are plentiful, the ETC becomes more reduced and electrons from complexes I and III can be donated directly to O₂ to generate superoxide anion (O₂.⁻), the first of the ROS. O₂.⁻ is converted to H₂O₂ by mitochondrial manganese superoxide dismutase (MnSOD), and H₂O₂ can be converted to water by glutathione peroxidase (GPx) or catalase. H₂O₂ can also acquire an additional electron from a reduced transition metal to generate the highly reactive hydroxyl radical (.OH). H₂O₂, which is semi-stable, can also diffuse out of the mitochondrion into the cytosol and the nucleus where is can act²⁷.

At high levels ROS are toxic, but at low levels they are mitogenic, presumably though interacting with various nuclear regulatory factors (AP-I, NF-κB APE/ref-1)²⁸, regulatory kinases (Src kinase, protein kinase C, MAPK), receptor tyrosine kinases²⁹, protein-tyrosine phosphatases³⁰ and angiogenic factors^{31,32}. Consistent with mitochondrial ROS being important in tumor formation, MnSOD is reduced in many types of tumors including prostate cancer, mutations in the MnSOD gene promoter have been observed in a number of tumors, and transformation of certain tumors with the MnSOD cDNA can reverse the malignant phenotype^{29,33-35}.

The mitochondrion contains a genome without introns that encodes the machinery of protein production including a 12S ribosomal RNA (rRNA), 16S rRNA, and a complete complement of transfer RNAs (tRNAs). Mitochondrial OXPHOS is assembled from multiple polypeptides, some encoded by the mitochondrial DNA (mtDNA) and others by the nuclear DNA (nDNA). In addition to the 12S and 16S rRNAs and 22 tRNAs for mitochondrial protein synthesis, the mtDNA encodes for 13 polypeptides, all of which are components of large protein complexes (Respiratory complexes I-V) localized in the inner mitochondrial membrane. These 13 polypeptides include seven (ND1, 2, 3, 4L, 4, 5, and 6) of the 46 polypeptides of complex I, one (cytochrome b) of the II polypeptides of complex III, three (cytochrome c oxidase, subunit I (COI), COII, and COIII) of the 13 polypeptides of complex IV and two (ATP6 and 8) of the 16 polypeptides of complex V. COI is the main catalytic subunit of cytochrome c oxidase (complex IV) and ATP6 is central to the proton channel of the ATP synthase (complex V)²⁷.

That inhibition of OXPHOS increases ROS production has been confirmed in mice in which the heart-muscle isoform gene of the ANT gene (Ant1) was inactivated. This resulted in the hyperpolarization of ΔP, increased ROS generation, and elevated mtDNA damage^{36,37}.

The “classic” mitochondrial diseases are caused either by mutations of the mtDNA or mutations of nuclear genes that code for peptides of the mitochondrial respiratory complexes. They affect primarily the neuromuscular system and include Leber hereditary optic neuropathy (due to mutations in mitochondrial components of respiratory complex (RC I), Leigh syndrome (a disease of the basal ganglia due to mutations in mitochondrial components of RC V), and approximately 10 other specific diseases³⁸. Some of the common features of these diseases are that they are characterized by progression with ageing, are due to inherited mtDNA mutations, and affect specific organs and organ systems despite being present in all cells.

The mitochondria are key players in cancer biology mediating apoptosis and serving as a common final checkpoint before the first committed step in this process, the release of cytochrome c. The susceptibility to apoptosis is gated at the mitochondria and is at least partially determined by the specific protein and lipid components of this organelle. VDAC1, a mitochondrial membrane protein, serves as a binding partner for various members of the Bcl family of proteins mediating their well-known influence on apoptosis³⁹. Furthermore, the mitochondria can be the specific target of anticancer therapies, can influence cellular oxidation-reduction (redox) status and also provide ATP for kinases and the regulation of cell cycle. Finally, the mitochondria are frequently altered as cancer cells become resistant to chemotherapy⁴⁰.

Previously, the dramatic, region-specific variation of the mtDNA sequence observed in indigenous populations around the world was thought to be due simply to genetic drift. A recent analysis of 1125 global mtDNA sequences showed that specific mtDNA replacement mutations permitted our ancestors to adapt to more northern climates, and that these same variants are affecting our health today. In other words, as humans evolved out of Africa, those mtDNA mutations (reflected in specific mitochondrial haplogroups) that allowed greater heat generation were selected for but have residual consequences in disease predisposition⁴¹. Thus haplogroup T is associated with Wolfram syndrome and asthenozoospermia while haplogroups J and K are linked to increased susceptibility to multiple sclerosis^{42,43}.

Screening tests are more useful when applied to high-risk groups than the general population. The European Randomized Study of Screening for Prostate Cancer (ERSPC) is a large, randomized controlled trial of screening versus control, conducted in eight European countries (Belgium, Finland, France, Italy, the Netherlands, Spain, Sweden, and Switzerland). In this study, the relationship between PSA levels and positive predictive value is high at the time of the first PSA test, but is low in subsequent tests because of the lower incidence of disease in patients receiving multiple tests⁴⁴. This finding may lead patients and physicians to stop screening after an initially negative serum PSA. While this may be appropriate for the general population, high-risk populations may benefit from continued serial screening. Another reason to identify high-risk groups is that they may be appropriate for screening starting at an earlier age. The American Cancer Society's current screening recommendations include earlier screening for individuals in currently accepted high-risk groups (those of African American ethnicity and those with a positive family history).

Methods are needed for easily and quickly identifying groups at high risk of developing cancers such as protate and renal cancers.

All publications referred to herein are incorporated by reference to the extent not inconsistent herewith.

### SUMMARY OF THE INVENTION

Inherited missense mutations in mitochondrial genes, and individuals classed in specific haplotypes, are found at a disproportionately high rate in prostate cancer patients compared to controls. Identifying individuals at increased risk of developing prostate cancer through a simple genetic test such as provided herein aids in developing efficient screening, early detection, and prevention strategies for this disease, all of which are more effective when instituted in at-risk populations rather than the general population.

We have discovered that inherited missense point mutations in the mitochondrially-encoded cytochrome c oxidase subunit 1 (COI) gene occur in 12% of patients with prostate cancer compared to less than 2% of controls. We have also discovered that individuals with a specific mitochondrial haplotype (haplogroup U) constitute a high-risk group for the development of prostate cancer.

A method is provided for identifying a subject likely to have, or at risk of developing a disease correlated with increased reactive oxygen species (ROS), such as cancer, said method comprising identifying in said subject a missense mutation in a nucleic acid of Complex III, IV and/or V of the OXPHOS system. The subject can be a human or other animal, preferably a human. The term “likely to have cancer” means having a greater than 10% chance of having cancer, or greater than a 50% chance of having cancer, or greater than a 75% chance of having cancer. The term “at risk of developing cancer” means having a greater than 10% chance of developing cancer, or greater than a 50% chance of developing cancer, or greater than a 75% chance of developing cancer.

Such mutations can also be identified in subjects suffering from a neuromuscular disease associated with such mitochondrial mutations; however, surprisingly, they have also been identified in subjects who have cancer or are likely to develop cancer, and who do not exhibit a neuromuscular disease.

Such mutations are associated with and can be diagnostic of any type of cancer, for example, a cancer selected from the group consisting of colon cancer, lung cancer, kidney cancer, breast cancer, and prostate cancer. Specific mutations identified herein are strongly identified with prostate cancer. Mutations of a cytochrome oxidase gene, e.g., the COI gene, are particularly associated with prostate cancer.

Such specific missense mutations include: C5911T, G5913A, A5935G, G5949A, G5973A, G6081A, G6150A, T6124C, T6253C, G6261A, G6267A, G6285A, C6340T, G6480A, A6663G, G6924T, G7041A, T7080C, A7083G, A7158G, A7305C, A14769G, and C8932T. Such mutations can also include G5949A, A14769G, and C8932T, as well as T7389C. Any group of the foregoing mutations, alone or combined with other OXPHOS system mutations, can be indicative a cancer or predisposition to cancer. The mutations referred to herein are named by a first letter indicating the nucleotide present at the position indicated by the numerals following this first letter in the Cambridge mitochondrial sequence (Genbank No. J01415), followed by a last letter indicating the nucleotide of the Cambridge sequence that has been replaced by the nucleotide indicted by the first letter of the mutation name.

Identification of a single mutation is often sufficient to identify the likelihood of a patient having cancer, having a predisposition to cancer, or having a likelihood of passing on a predisposition to cancer to descendants. In other embodiments, at least two of said mutations are identified. In still other embodiments, at least three of said mutations, or any number up to all of the specific mutations identified herein as associated with cancer are identified. Mutations having the effect of inhibiting oxidative phosphorylation (OXPHOS) and increasing reactive oxygen species (ROS) can be used in the methods of this invention to predict or identify cancer.

The methods of this invention include identifying such mutations in a sample derived from the subject. A sample derived from a subject, as known to the art, can be any bodily fluid or tissue, including tumor and non-tumor tissue. The sample can contain nucleic acids such as DNA, RNA, or cDNA, or proteins or polypeptides resulting from expression of nucleic acid coding sequences containing the missense mutations. The sample can be tissue, blood, urine, cerebral spinal fluid (CSF), sputum, semen, cervicovaginal swab, intestinal wash, tumor tissue or other sample known to the art containing nucleic acids or gene expression products. Peripheral blood samples are preferred, and lymphocytes are especially preferred. The sample derived from the patient which is tested in the method of this invention may be a fluid or fraction extracted from the original sample taken from the patient, or a portion of the original sample. The sample derived from the patient may also be a cell culture of the patient's cells, or of hybrid cells constructed from the patient's cells or cybrid cells as described herein.

Detection of missense mutations can be performed by any means known to the art including detection of altered gene expression products or detection of altered nucleic acids. Detection of missense mutations in nucleic acids includes contacting the sample with an array comprising nucleic acid sequences for identifying missense mutations. Arrays are described, e.g., in PCT Patent Publication No. WO 03/020220, “Mitochondrial Biology Expression Arrays,” Wallace, Douglas C. et al., inventors, published Mar. 13, 2003, incorporated herein by reference to the extent not inconsistent herewith. Arrays including large numbers of probes, such as the human MITOCHIP described in said PCT publication, may be used, or arrays containing only probes capable of identifying mutations in OXPHOS system genes may be used. Preferably the gene is a Complex IV or V gene, more preferably a cytochrome oxidase I (COI) gene.

Subjects who have been identified by the methods of this invention as likely to have, or at risk for developing, cancer can then be counseled on cancer prevention and management, including counseling on obtaining frequent checkups and self-examination. Further tests, including periodic follow-up tests, for cancer can also be undertaken, or in the event cancer is identified, suitable treatment can be instituted.

This invention also provides a method for detecting in a subject a condition selected from the group consisting of: likelihood of having cancer, being at risk of developing cancer, and likelihood of passing a predisposition to cancer to progeny, comprising identifying in non-tumor tissue of said subject a missense mutation in a gene of Complex III, IV and/or V of the mitochondrial OXPHOS system in non-tumor tissue of said subject. The cancer may be any cancer, as set forth above. In one embodiment, the cancer is prostate cancer. The mutation may be a nuclear or mitochondrial mutation. When the mutation is homoplasmic in non-tumor tissue this is an indication it is an inherited and inheritable trait, and that the subject is likely to pass on the mutation to her progeny in the case of mutations in mitochondrial DNA, or his or her progeny in the case of mutations in nuclear DNA. Both homoplasmic and heteroplasmic mutations in non-tumor tissue can indicate the presence of prostate cancer. When the subject is a human male, and such mutations are detected, further testing for the presence of prostate cancer may be undertaken, as known to the art.

Specific mutations for this purpose include the following missense mutations of the mitochondrial COI gene: C5911T, G5913A, A5935G, G5973A, G6081A, G6150A, T6124C, T6253C, G6261A, G6267A, G6285A, C6340T, G6480A, A6663G, G7041A, T7080C, A7083G, A7158G, A7305C, and T7389C. These mutations are expressed in relation to the published Cambridge mitochondrial sequence, Genbank Sequence J01415. The first letter is the nucleotide appearing at the position designated by the numbers following that letter in the Cambridge sequence, and the last letter is the nucleotide to which the Cambridge sequence nucleotide has been changed to produce the missense mutation.

This invention also provides a method for detecting a heritable predisposition to cancer in a subject comprising detecting in non-tumor tissue of said subject, a homoplasmic missense mutation in a gene of the OXPHOS system. The type of cancer may be any type known to the art as discussed above, e.g. prostate cancer.

This invention also provides a nucleic acid array consisting essentially of probes made of normal sequences from the OXPHOS system, or only complexes III, IV and V thereof, or only one or two of said complexes, and selected missense mutations thereof. The term “selected mutations” thereof means that only missense mutations are included, and that the array does not include all possible mutations at all possible locations. The term “consisting essentially of” in this context means that only probes from the OXPHOS system are included, or with respect to arrays comprising only probes from sequences of certain complexes, only sequences from genes of those complexes are included. Probes suitable for use in such arrays are commercially available, e.g., as described in PCT Publication No. WO 03/020220.

A useful subset of such probes includes probes comprising the following mutations C5911T, G5913A, A5935G, G5973A, G6081A, G6150A, T6124C, T6253C, G6261A, G6267A, G6285A, C6340T, G6480A, A6663G, G7041A, T7080C, A7083G, A7158G, and A7305C.

The arrays of this invention may be part of a system including means for reading and analyzing the results of hybridization of sample components to the array, such additional components being known to the art, for example as described in PCT Publication No. WO 03/020220.

### BRIEF DESCRIPTION OF THE DRAWINGS

FIG. 1. Tumor 18 COI G16X Mutation in LCM-isolated prostate cancer epithelium. A. mtDNA sequencing chromatograms of LCM-collected pure prostate cancer epithelium (upper chromatogram) reveals only the mutant 5949T (G5949A, opposite strand) mtDNAs and pure normal epithelium (lower chromatogram) reveals only the wildtype 5949C (G5949G, opposite strand) mtDNAs; B. RFLP analysis of the G5949A mutation detected through the creation of a new Ddel restriction site. Lanes 1 to 4, Ddel digests. Lane 1=pure cancer epithelium (pure mutant), lane 2 pure normal epithelium (pure wild type), Lanes 3 and 4=wild type controls. Lane 5=molecular weight markers; C. Immunohistochemical staining of a section from Tumor 18 stained with a COI-specific antibody: M=malignant glands, N=the normal glands.

FIG. 2. Increased Tumor Growth of PC3(mtDNA T8993G versus T) Cybrids.

FIG. 3. Dihydroethidium evaluation of PC3(mtDNA T8993T versus G) tumor ROS production. A. Relative fluorescence level seen in PC3(mtDNA T8993T) tumor sections, representative of wildtype clones 3 and 10; B. Relative fluorescence level seen in PC3(mtDNA T8993G) tumor sections, representative of mutant clones 5, 8, and 20.

FIG. 4. Cybrid Formation. A=Lymphoblast with mutant mtDNA. B=Cytoplast with mutation (enucleated lymphoblast). C=PC-3 prostate cancer cell line. D=Rhodamine-6-G treated PC-3 cell depleted of mitochondria. E=Cybrid from electrofusion of B and D yields cell with prostate cancer nucleus and desired mtDNA mutation. Cybrid E combines mutant mtDNA of A with prostate cancer nucleus of C.

FIG. 5. In vitro proliferation of T8993G mutant cybrid line showing 50% greater proliferation of Mutant line (M).

FIG. 6. Baseline apoptotic index of 78993G mutant cybrid line; A. by DNA laddering; B. by Annexin V/Flow.

FIG. 7. In vitro Reactive Oxygen determination.

FIG. 8. Correlation of haplogroups with: A. The U haplogroup is significantly overrepresented in the prostate cancer patients (p=0.019); B. The U haplogroup is significantly overrepresented in the renal cancer patients (p=0.005) while the T haplogroup is significantly underrepresented (p=0.013).

### DETAILED DESCRIPTION OF THE INVENTION

An individual's inherited mitochondrial genotype predisposes to, or protects patients from, the development of certain cancer types and this is reflected in different frequencies of mitochondrial genotypes in cancer patients compared to controls.

We have demonstrated that germ-line (inherited) mutations of the COI gene appear to be over-represented in prostate cancer patients⁴⁵. Furthermore, the vast majority of mtDNA mutations present in the cancer are in fact inherited and present in all cells of the body, a fact that is not appreciated when analysis is performed only on tumor tissues.

When examining disease-associated family pedigrees, X-linked transmission can look very much like mitochondrial inheritance because of the matrilineal inheritance of the mitochondria and mtDNA. Epidemiological studies have documented excess risk of prostate cancer in men with affected brothers compared to those with affected fathers⁴⁶. This recognition has led to the identification of some families demonstrating linkage to the Xq27-28 locus⁴⁷. Not all maternal pedigrees show linkage to this or any other X-locus. The increased brother-brother concordance of disease over father-brother that is not accounted for by X-linkage can be attributed to mitochondrial inheritance.

The “classic” mitochondrial diseases are caused either by mutations of the mtDNA or mutations of nuclear genes that code for peptides of the mitochondrial respiratory complexes. They affect primarily the neuromuscular system and include Leber hereditary optic neuropathy (due to mutations in mitochondrial components of respiratory complex (RC) I), Leigh syndrome (a disease of the basal ganglia due to mutations in mitochondrial components of RC V), and approximately 10 other specific diseases³⁸. Some of the common features of these diseases are that they are characterized by progression with ageing, are due to inherited mtDNA mutations, and affect specific organs and organ systems despite being present in all cells.

The mitochondria are key players in cancer biology mediating apoptosis and serving as a common final checkpoint before the first committed step in this process, the release of cytochrome c. The susceptibility to apoptosis is gated at the mitochondria and is at least partially determined by the specific protein and lipid components of this organelle. VDAC1, a mitochondrial membrane protein, serves as a binding partner for various members of the Bcl family of proteins mediating their well-known influence on apoptosis³⁹. Furthermore, the mitochondria can be the specific target of anticancer therapies, can influence cellular oxidation-reduction (redox) status and also provide ATP for kinases and the regulation of cell cycle. Finally, the mitochondria are frequently altered as cancer cells become resistant to chemotherapy⁴⁰.

Previously, the dramatic, region-specific variation of the mtDNA sequence observed in indigenous populations around the world was thought to be due simply to genetic drift. A recent analysis of 1125 global mtDNA sequences showed that specific mtDNA replacement mutations permitted our ancestors to adapt to more northern climates, and that these same variants are affecting our health today. In other words, as humans evolved out of Africa, those mtDNA mutations (reflected in specific mitochondrial haplogroups) that allowed greater heat generation were selected for but have residual consequences in disease predisposition⁴¹. Thus haplogroup T is associated with Wolfram syndrome and asthenozoospermia while haplogroups J and K are linked to increased susceptibility to multiple sclerosis^{42,43}.

Mutations in the mtDNA have been found to fulfill all of the criteria expected for pathogenic mutations causing prostate cancer. Focusing on the COI gene, we found that 11-12% of all prostate cancer patients harbored COI mutations that altered conserved amino acids [mean conservation index (CI)=83%], while <2% of no-cancer controls and 7.8% of the general population had COI mutations, the later altering less conserved amino acids [CI=71%]. Four conserved prostate cancer COI mutations were found in multiple independent patients on different mtDNA backgrounds. Three other tumors contained heteroplasmic COI mutations, one of which created a stop codon. This later tumor also contained a germline ATP6 mutation. Thus, both germline and somatic mtDNA mutations contribute to prostate cancer. Many tumors have been found to produce increased reactive oxygen species (ROS), and mtDNA mutations that inhibit oxidative phosphorylation (OXPHOS) can increase ROS production and thus contribute to tumorigenicity. To determine if this was the case, we introduced the pathogenic mtDNA ATP6 T8993G mutation into the PC3 prostate cancer cell line through cybrid transfer and tested for tumor growth in nude mice. This mutation changes the highly conserved leucine at amino acid 156 to an arginine, resulting in a proton channel defect and a 70% decrease in hyperpolarization of the mitochondrial transmembrane electrochemical gradient (Δψ), the secondary inhibition of the electron transport chain and the reduction of electrons from the electron transport chain to molecular oxygen to give superoxide anion and H₂O₂. The resulting mutant (T8993G) cybrids were found to generate tumors that were 7 times larger than the wildtype (T8993T) cybrids, while the wildtype cybrids barely grew in the mice. The mutant tumors also generated significantly more ROS. Therefore, mtDNA mutations do play an important role in the etiology of prostate cancer.

The possibility that a mitochondrial contribution to tumorigenicity could be increased ROS production was suggested by the observation that the mev-1 mutation in the SDHC gene of C. elegans markedly increases ROS production^{48,49} and increased ROS production has been proposed to be an important factor in tumor formation in association with inactivation of p16ink4a and p53²⁴. If mitochondrial dysfunction and ROS production contribute to cancer, then this would make two predictions. First, cancers should harbor both germline and somatic mtDNA mutations, which should partially inhibit OXPHOS and thus increase ROS production. Second, mtDNA mutations that increase ROS production should stimulate tumor growth. We have tested both of these predictions on prostate cancer and confirmed their validity. Therefore mitochondrial defects contribute to the etiology of cancer.

Unique sets of mitochondrial DNA polymorphisms define the ancestral origin, or haplogroup, of each person's mitochondrial DNA⁵⁰. Patients with certain mitochondrial haplogroups are more susceptible to nonmalignant mitochondrial diseases (e.g. Leber's hereditary optic neuropathy, Alzheimer's, multiple sclerosis, Parkinson's disease)⁵¹⁻⁵³. Because of the central role of mitochondria in regulating apoptosis, we hypothesized that an individual's mitochondrial haplogroup may either protect against or predispose to the development of prostatic and renal carcinomas. One prediction of this hypothesis is that mitochondrial lineage represented by mtDNA haplogroup is different in cases and controls. It was the purpose of this study to define the mitochondrial haplogroups of a group of Caucasian prostate and renal cancer patients and compare these to a regional (Georgia) North American Caucasian population control, thereby testing this prediction and hypothesis. Our results showed that prostate cancer is associated with haplogroup U.

### EXAMPLES

### Materials and Methods

### Patient Materials:

All patient studies were implemented under Emory University IRB approved protocols. Histologically confirmed prostate cancer samples were selected from our collection of radical prostatectomies, institutional tissue resources, and microdissected samples prepare between 1995 and 2002. The “no-cancer” control group was assembled from subjects that had undergone prostate biopsy and been found to be free of prostate cancer. These individuals were all at least 50 years old and had a PSA <4 ng/ml.

Sequencing the mtDNA COI Gene:

In order to determine which (if any) area of the mitochondrial genome was mutated in prostate cancer we began our investigations by sequencing the entire genome in multiple prostate cancer cases. It rapidly became apparent that the gene most frequently affected with missense mutations was cytochrome c oxidase subunit 1 (COI). For this reason the majority of our subsequent sequencing effort has concentrated on this gene, with positive results.

At the time we began our investigations, mtDNA mutations in cancer were thought to be exclusively somatic. No one had yet demonstrated germ line mutations in cancer patients. We therefore began by sequencing laser capture microdissected cancer epithelium but when we sequenced the adjacent normal epithelium we found that these cells almost always contained the same mutations. In order to determine whether the mutations were inherited we next sequenced the COI gene from buffy coat DNA (peripheral lymphocytes) and again found that they almost always contained the same mutations, confirming germ line transmission.

The mtDNA region encompassing COI was amplified using a forward primer starting at nucleotide pair (np) 5772 (5′ AGG TTT GM GCT GCT TCT TC 3′) (SEQ ID NO:1] and a reverse primer ending at np 7600 (5′ CGC TGC ATG TGC CAT TAA GA 3′) [SEQ ID NO:2]. Both strands of the COI PCR product were cycle sequenced using the slip primers in the forward direction starting at np 6080 (5′ TCT ACA ACG TTA TCG TCA CA 3′) [SEQ ID NO:3] and at np 6930 (5′ TGC AGT GCT CTG AGC CCT AG 3′) [SEQ ID NO:4] and in the reverse direction starting at np 6340 (5′ CTA GGT GTA AGG AGA AGA TG 3′) [SEQ ID NO:5] and at np 7150 (5′ GAT TTA CGC CGA TGA ATA TG 3′) [SEQ ID NO:6]. The templates were denatured at 96° C. and primers extended in the presence of “Big Dye Terminators” for 25 cycles of 96° C. for 10 sec, then 55° C. for 5 sec, and 60° C. for 4 min. The reactions were chilled to 4° C., and the excess dye terminators removed by Centri-Sep Columns. The trace files were determined using an ABI Prism 3100 genetic analyzer, analyzed using Sequencher gene analysis software v 4.1 (Gene Codes, Ann Arbor, Mich.) and interpreted within the context of MITOMAP (http://www.mitomap.org) and our current collection of 1338 complete mtDNA sequences⁵⁴. To assure that the sequence variants found were not due to the spurious amplification of nDNA pseudogenes, we scanned our mtDNA pseudogene data base⁵⁵ for any pseudogene that might have been amplified by the primers used. Only 1 (Emb/AL359496.30/on chromosome 6) matched the primers used and could have potentially been amplifiable in the above experiments. However, this pseudogene could not have contributed to the current results since it lacks all of the COI mutations that we found in the prostate cancer samples.

Histopathological and molecular analysis of LCM-isolated prostate cancer epithelium

Normal and tumor prostate epithelial cells were collected by Laser Capture Microdissection (LCM). Fresh radical prostatectomy specimens, embedded in Tissue Tek OCT compound (Sakura Finetek, Torrance, Calif.), was frozen at −80° C. and sectioned by cryostat (Shandon Lipshaw, Pittsburgh, Pa.) to yield 7 μm sections. Each section was fixed in 70% alcohol and H&E stained⁵⁶. Prior to microdissection, cut sections were dehydrated in graded alcohols followed by xylene and air-dried for 5 min. Benign and malignant epithelial regions were selected by a pathologist and at least 5000 cells each were collected by LCM using a Pixcell II LCM system from Arcturus Engineering (Mountain View, Calif.).

DNA was extracted from the microdissected tissue by transfer to 0.04% proteinase K, 10 mM Tris-HCL (pH 8.0), 1 mM EDTA, and 1% Tween-20 and digested over night. Organic (phenol/chloroform) extraction was followed by ethanol/sodium acetate precipitation. DNA pellets were suspended in distilled water.

The entire mtDNA of Tumor 18 was amplified in 1 to 2 kb overlapping fragments, the fragments purified using Centricon-100 concentrator columns and cycle sequenced. The dried sequencing reactions were resuspended, heated at 94° C. for 2 min, loaded on “Long Ranger” DNA sequencing gels, and the sequencing traces determined using an ABI Prism 377 automated DNA sequencer.

The heteroplasmy of the COI G5949A mutation was analyzed by RFLP analysis. The mutant region was PCR amplified using a forward primer in which an A at np 5946 was changed to C, bold face (5′-CTCTACAAACCACAAAGACCTT-3′) [SEQ ID NO:7]. The combination of the patient's G5949A mutation plus the introduced C generates a new Ddel restriction site.

The presence of the COI polypeptide in prostate Tumor 18 tissue was analyzed by immunohistochemistry. Five micron, formalin-fixed, paraffin-embedded sections from tissue adjacent to the frozen section block used for LCM-directed DNA sequencing were deparaffinized and rehydrated, then steamed in citrate buffer (pH6) for 20 minutes and cooled for 10 minutes. After exposure to 3% hydrogen peroxide for 5 minutes at room temperature, mouse monoclonal anti-human COI antibody (Molecular probes, Eugene, Oreg.) at a dilution of 1:250 was reacted for 25 minutes. Then a biotinylated secondary linking antibody was reacted for 25 minutes, followed by a streptavidin bound peroxidase enzyme complex for 25 minutes. The sections were then stained with diaminobenzidine as chromogen for 5 minutes and counterstained with hematoxylin for 1 minute. Between incubations, the sections were washed with Tris-buffered Saline.

PC3(mtDNA T8993T/G) Cybrids:

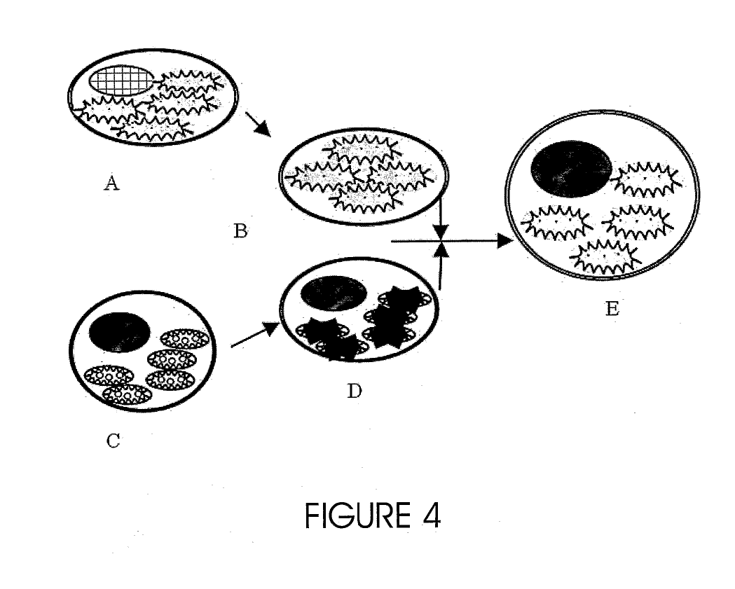

Transmitochondrial cybrids (FIG. 4) were prepared by treating PC3 prostate cancer cells with 5 μg/ml rhodamine 6-G (R6G) in culture medium for 7 days to cure them of their resident mitochondria⁵⁷. Then 3×10⁶ of the R-6G-treated PC3 cells were fused by electric shock to approximately 2×10⁷ cytoplasts obtained by Ficoll-cytochalasin B step gradient enucleation of homoplasmic mutant (T8993G) or homoplasmic wildtype (T8993T) 143B(TK⁻) cell lines⁵⁷. The homoplasmic T8993G mutant or T8993T wildtype donor cells were cybrids that had been previously prepared by fusing 143B(TK7) cells devoid of mtDNA (ρ⁰ cells) with cytoplasts from the lymphoblastoid cell line of a patient that was heteroplasmic for the mtDNA NARP/Leigh ATP6 T8993T/G mutation⁵⁸. R-6G PC3 cell to T8993G/T cytoplast fusions were induced in a BTX PN453 slide chamber with a 3.2 mm electrode gap using a BTX Electro Cell Manipulator 200 equipped with an Optimizer (Biotechnologies and Experimental Research, San Diego, Calif.). The fusion mixture was plated in DMEM containing 10% fetal calf serum, 4.5 mg/ml glucose, 1 mM pyruvate, and HAT (hypoxanthine-aminopterin-thymidine), but without uridine⁵⁷. The PC3 nuclear origin of the PC3(mtDNA T8993T/G) cybrid clones was confirmed using the insulin gene variable number tandem repeats⁵⁹ and the chromosome number confirmed by karyotyping. The mtDNA genotype was confirmed by HpaII digestion⁵⁷.

In vivo Tumorigenesis of PC3(mtDNA T8993T/G) Cybrids:

All animal experiments were carried out as part of an IACUC approved protocol. Six week old male athymic (nude) mice were purchased from Harlan Labs (Indianapolis) or Charles River (Wilmington, Mass.) and injected subcutaneously over the scapula with 10⁶ viable cybrid cells. Animals were maintained in sterile housing, 4 animals to a cage, and observed on a daily basis. At ten day intervals, the tumors were measured using calipers and the volumes of the tumors calculated using V=(L×W²)/2. Animals were euthanized with carbon monoxide.

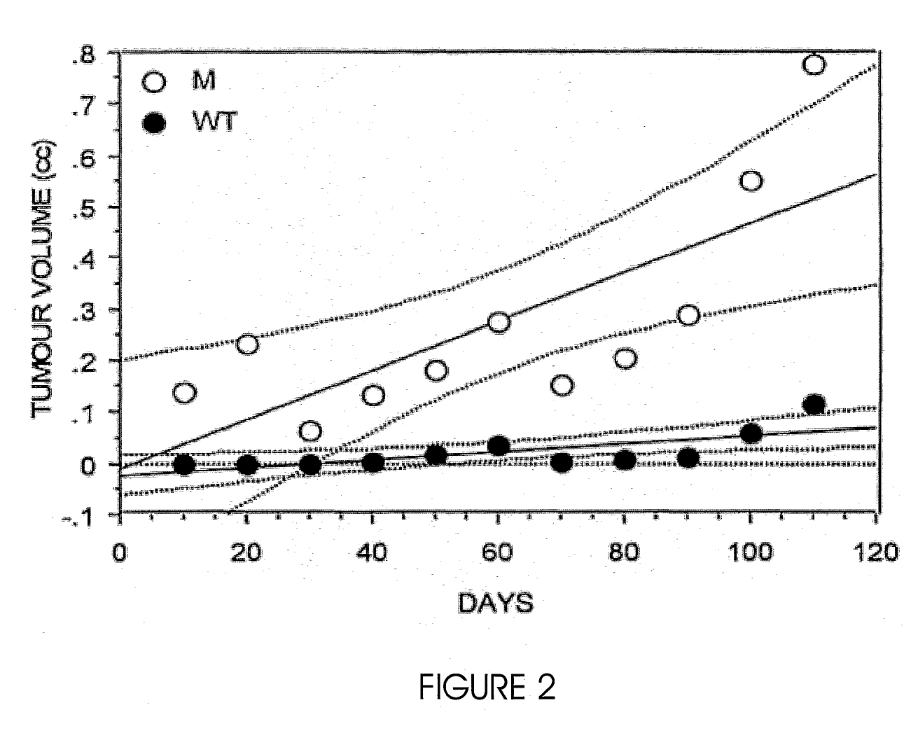

FIG. 2 is a composite of four independent experiments in which nude mice were injected subcutaneously with six different mutant PC3 cybrid clones [PC3(mtDNA T8993G)] and four different wildtype cybrid clones [PC3(mtDNA T8993T)]. All six mutant and four wildtype clones were tested at an early passage (P˜6) and two of the mutant clones and one wildtype clone were again tested at a later passage (P˜25). A total of 91 animals were injected with the mutant clones, 71 with the early passage and 20 with the late passage clones; while 55 mice were injected with the wildtype clones, 45 with the early passage and 10 with the late passage. Each injected mouse was measured for tumor volume every ten days, with the final data set encompassing 11 time points. However, in experiments where the animals receiving the mutant clones developed debilitating tumors, the mice had to be euthanized prior to the maximum experimental time point. This resulted in a final total of 615 determinations of tumor volume for mice harboring the mutant clones and 378 determinations for those harboring the wildtype clones.

ROS Production in Tumors:

Hydroethidium (HEt) was obtained from Molecular Probes (Eugene, Oreg.) and a 10 mM stock was prepared in dry N₂-sparged dimethylsulfoxide (DMSO), packed under N², and stored at −80° C. Working stocks consisted of 1 uM dilutions made in HBSS using fresh aliquots for each experiment. Tumor slices were obtained from masses generated from the in vivo comparison of tumorigenesis of mutant and wild type containing tumor cells subcutaneously implanted in nude mice. The tumors were dissected out rapidly, placed in OCT compound in plastic holding cassettes and flash-frozen in a methylbutane chilled in liquid nitrogen. Microtome slices (30 um) were collected and transferred on ice to the darkroom. Slices were treated with HEt for 30 min at 37° C. in a humidified 5% CO2 incubator. Samples were analyzed via confocal microscopy utilizing an argon laser at 510 nm excitation with 595 nm emission. The effect of the 8993G mutation on ROS production in tumors in vivo was assayed by hydroethidium oxidation⁶⁰. Fresh frozen xenograft sections containing intact tumor cells were treated with hydroethidium that intercalates in nuclear DNA and fluoresces only after reaction with intracellular superoxide. The prostate cancer cells harboring the mutant 8993G mtDNA showed substantially higher fluorescence (clones 5, 8, and 20) than the cancer cells harboring the normal 8993T mtDNA (Clones 10 and 3). Thus, the 8993G mutation also increased cancer cell ROS (superoxide) production in vivo when compared to tumors that differ by only one base in the mtDNA.

Results

Identification of the mtDNA Variants in LCM Isolated Prostate Cancer Epithelium.

To determine if mtDNA mutations were associated with the prostate cancer, we used LCM to isolate prostate cancer epithelial cells from several prostate tumors and sequenced segments of their mtDNAs. This revealed a variety of potentially pathogenic mtDNA mutations. As an example, for Tumor #18, the entire mtDNA was sequenced in a series of overlapping segments. This revealed 38 base substitutions relative to the Cambridge Reference Sequence (MITOMAP, http://www.mitomap.org) including 31 previously reported polymorphisms and 7 new mutations; the later including 1 ribosomal RNA mutation and 3 missense mutations. The three new amino acid substitution mutations included a chain termination mutation in COI (G5949A) and two missense mutations, one in cytb (A14769G) and the other in ATP6 (C8932T).

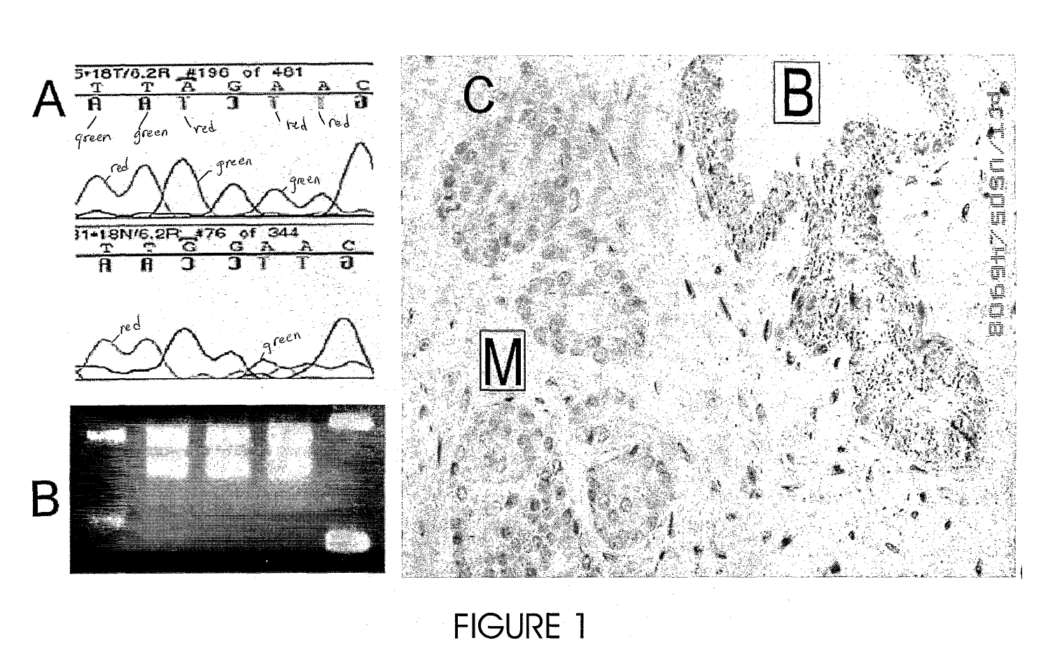

The G5949A mutation introduced a stop codon into COI at amino acid 16 (G16X) (FIG. 1A). To determine the origin of this mutation, we developed a RFLP test for the mutation and tested the cancerous and normal epithelium from this prostate. This revealed that the cancerous epithelial cells of Tumor #18 were homoplasmic mutant while the adjacent normal epithelial cells were homoplasmic wildtype (FIG. 1B). Hence, this mutation must have arisen during the genesis of the cancer cell and then segregated to pure mutant in the malignant cells.

To determine if the G16X mutation actually eliminated the COI protein from the prostate cancer cells, we performed immunohistochemistry on tumor sections (FIG. 1C). The normal epithelial cells proved to be strongly positive for COI while the adjacent cancer cells were completely negative.

The cytb A14769G mutation in this patient altered an amino acid (N8S) with a relatively low interspecific amino acid conservation (CI)=20.5%, indicating that this variant probably had limited effect on the cellular physiology. By contrast, the ATP6 C8932T mutation altered an amino acid (P136G) with a CI=64%, which could be functionally significant. Therefore, both germline and somatic mtDNA mutations may have contributed to the formation of Tumor #18.

COI Mutations in Prostate Cancer

The loss of the mtDNA encoded COI subunit in Tumor 18 is consistent with proteomic surveys of LCM isolated prostate cancer epithelia which revealed that the ratio of nDNA encoded complex IV subunits (COX IV, Vb and Vlc) to mtDNA-encoded subunits (COI and 11) was increased in most prostate tumors^{61,62}. Hence, deficiency of mtDNA COI, II, and III subunits might be a common feature of prostate cancer.

To determine if this was true, we sequenced the COI genes from multiple prostate cancer tumors and controls. We chose to study only the COI genes because this permitted us to survey a large number of tumor and control samples, thus making statistical evaluation feasible. Moreover, COI mutations have been observed in the mtDNAs in colon cancer cell lines (2), colonic crypt cells⁶³, and sideroblastic anemia patients^{64,65}. However, polymorphisms and pathogenic mutations in COI are relatively uncommon (MITOMAP, http://www.mitomap.org)^{54,66}.

DNA was extracted and the COI gene sequenced from prostatectomy specimens or peripheral blood cells taken from 260 European and African American patients with pathologically confirmed prostate cancer and from the lymphocytes of 54 “no-cancer” (prostate cancer negative) controls. COI missense mutations (Table 1) were found in 12% of the prostate cancer patients, but in only 1.9% of the no-cancer controls, a significant increase in frequency (P=0.023) (Table 2). Furthermore, in a population sample of 1019 European and African mtDNA sequences, 7.8% had COI mutations, which was also significantly lower than that of cancerous prostates (P=0.015) (Table 2). Since COI missense polymorphisms are more common in African mtDNAs of macro-haplogroup L than in the rest of the world⁶⁶, we also analyzed only patients and controls of European ancestry. From this group, COI missense mutations were found in 11% of the prostate cancer specimens, 0% of no-cancer controls (P=0.016), and 6.5% in a population sample of 898 Europeans (P=0.025) (Table 2). Thus COI mutations are significantly increased in prostate cancer over the no-cancer controls and the general population.

The interspecific conservation of the altered COI amino acids in prostate cancer was also significantly higher than that in the general population. The average CI for the prostate cancer mutations was 83±25%, while that for a general population sample of 1338 mtDNA sequences was 71±35 (P=0.029). The CI of the prostate cancer COI mutations was comparable to the CI observed for global human mtDNA “adaptive mutations” (85±9%), and far above the CI of global “neutral polymorphisms” (23±15%)54. Thus, the prostate cancer COI mutations must be functionally significant.

Three of the prostate cancer COI mutations had the characteristics of new somatic pathogenic mutations, being heteroplasmic and changing highly conserved amino acids. The first of these mutations was the Tumor 18 chain termination mutation, G5949A (G16X). The second was a T6124C mutation (M74T) with a CI=95%, that was heteroplasmic in both the prostate tissue and blood cells. The third C6924T (A341S) with a CI=100%, was primarily mutant in the prostate tissue but wild type in blood (Table 1).

Four other prostate cancer COI mutations were found in more than one patient and in each case were associated with prostate cancer. The T6253C mutation (CI=69%) was found in three independent cases, all on haplogroup H, the most common European haplogroup. The C6340T mutation (CI=79%) was observed in two patients on two different haplogroup backgrounds (H and N). The G6261A mutation (CI=97%) was observed in six patients on four different haplogroups (J, T, L1, and N). Finally, the A6663G mutation (CI=95%) was observed in five patients on two different haplogroups (L2 and unclassified) (Table 1).

Since the T6253C, C6340T, G6261A and A6663G mutations were homoplasmic in these patient's lymphocytes, they must have arisen in the female germline. That germline mutations are important in prostate cancer was supported by the observation that none of the European descent no-cancer control men had COI mutations, yet 6.5% of the European population had COI mutations and 11% of the European prostate cancer patient men had COI mutations. Since the frequency of COI mutations is significantly different between the non-cancer controls and the general population (P=0.05) and between the general population and the prostate cancer positive men (p=0.016), it follows that men that harbor germline COI mutations must have a substantially increased risk of developing prostate cancer. Therefore, both somatic and germline COI mutations are associated with prostate cancer, and COI mutations must be a significant risk factor for prostate cancer.

We have exhaustively surveyed COI mutations in this study. Additional mtDNA polypeptide mutations can contribute to the etiology of prostate cancer. This is supported by the ATP6 C8932T (P136C) observed in Tumor 18 as well as the presence of two novel missense mutations in the complete mtDNA sequence of the PC3 tumor cell line which is haplogroup U5 and contained a np T11120C (ND4, F121L, CI=12.8%) variant of unlikely functional significance and a np C13802T (ND5, T489M, CI=62.5%) variant that could be functionally relevant. Additional mtDNA missense mutations were found in other tumors analyzed from LCM material (data not shown).

Cybrid Studies Reveal that mtDNA Mutations Enhance Cancer Cell Growth

To investigate the functional importance of mtDNA mutations for prostate cancer, we chose to model the Tumor 18 germline ATP6 C8932T (P136C) mutation using the well characterized pathogenic ATP6 np 8993G L156R mutation⁶⁷. The ATP6 np 8993G mutation is just 20 amino acids away from the P136C mutation and is known to cause a 70% inhibition in ADP-stimulated respiration⁵⁸ and increased mitochondrial ROS production⁶⁸.

We introduced mtDNAs harboring the ATP6 mutant T8993G or wildtype T8993T base into a prostate cancer cell line through transmitochondrial cybrids. The mitochondrial donor cells were derived from the same heteroplasmic patient⁵⁸ and were homoplasmic for either the T8993G or T8993T mtDNA. These were enucleated and the cytoplasts fused to PC3 cells that had been cured of their resident mtDNAs using R-6G⁵⁷. Six PC3 cybrids that were homoplasmic for the mutant mtDNA (T8993G) [PC3(mtDNA T8993G) #s 2, 5, 8, 20, 21 and 26] and four cybrids that were homoplasmic wild type (T8993T) [PC3(mtDNA T8993T) WT#s 1, 3, 5 and 10] were isolated and studied further.

The PC3(mtDNA ATP6 T8993T) and PC3(mtDNA ATP6 T8993G) cybrids were injected subcutaneously into nude mice, in four separate experiments conducted on both early passage (˜P6) and late passage (˜P25) cultures. The results from all four experiments encompassing multiple trials for both the T8993G versus the T8993T clones were combined for each time point and the values averaged and plotted (FIG. 2). This revealed that the average tumor volume of the mutant PC3(mtDNA T8993G) cybrids was significantly higher than that of the wildtype PC3(mtDNA T8993T) cybrids at every time point (P<0.026 by Mann-Whitney test) (FIG. 2). Indeed, the PC3(mtDNA T8993T) wildtype cybrids barely grew at all in the mice. By day 110, the average tumor volume of the PC3(mtDNA T8993T) wildtype cybrids was 0.11 cc while that of the PC3(mtDNA T8993G) mutant cybrids was 0.78 cc, over a seven fold increase. Moreover, this is an underestimate of the differential growth rate of the PC3(mtDNA T8993G) tumors, since mice with rapidly growing tumors had to be euthanized throughout the various experiments. This removed the fastest growing PC3(mtDNA T8993G) tumors from the later average tumor size calculations, resulting in an uneven mutant cybrid curve with a reduced average slope (FIG. 2). Hence, the tumor growth rate of the PC3 prostate cancer cell nucleus was enhanced by the introduction of an ATP6 mutation known to reduce ATP synthase activity and increase mitochondrial ROS production⁵⁸⁶⁸

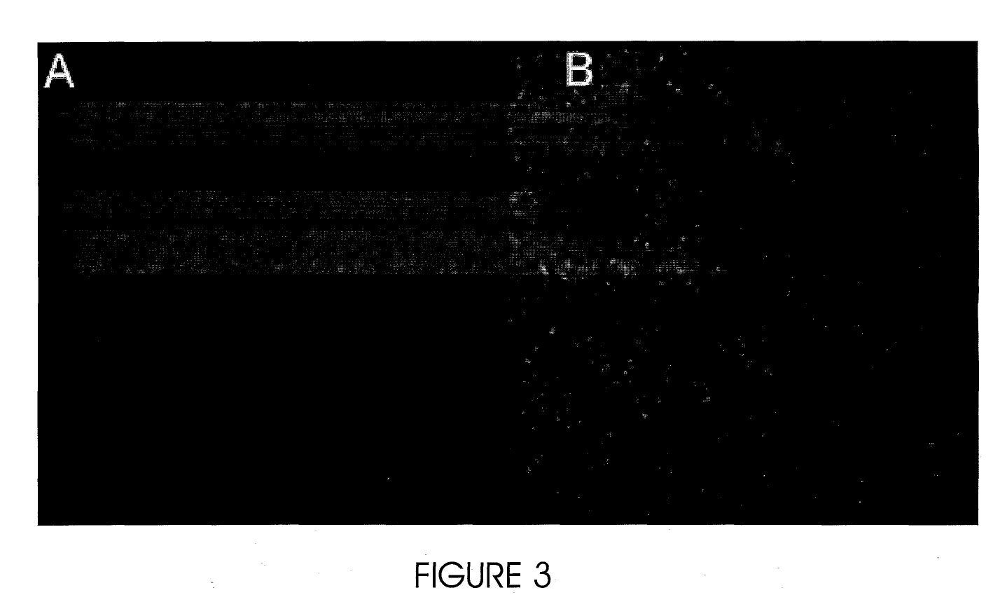

To confirm that the mutant PC3(mtDNA T8993G) cybrids did generate more ROS, we tested the tumor cells for superoxide anion production by staining tumor sections with DHE (FIG. 3). The non-fluorescent DHE is oxidized to fluorescent ethidium by O₂.⁻. The average fluorescence pixel density of the PC3(mtDNA T8993G) mutant tumor cells was 71.2±9.2 (N=3, MT5, 8, 20) while that of the PC3(mtDNA T8993T) wildtype tumor cells was 46.7±4.2 (N=2, WT3 & 10). Thus there was significantly more ROS produced by the PC3(mtDNA T8993G) mutant tumors (P=0.013 by T-test). Therefore, in prostate cancer cells that harbor mtDNA mutations which increase ROS production show increased tumor growth.

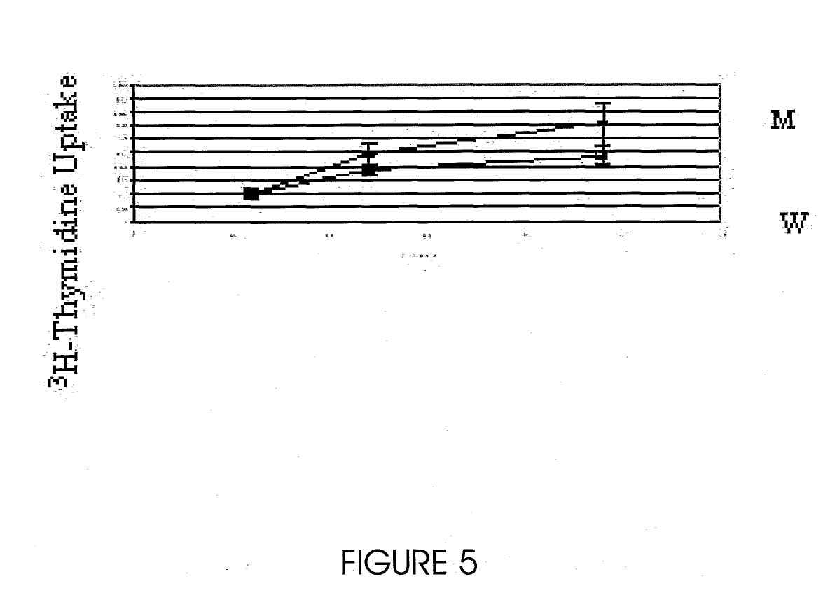

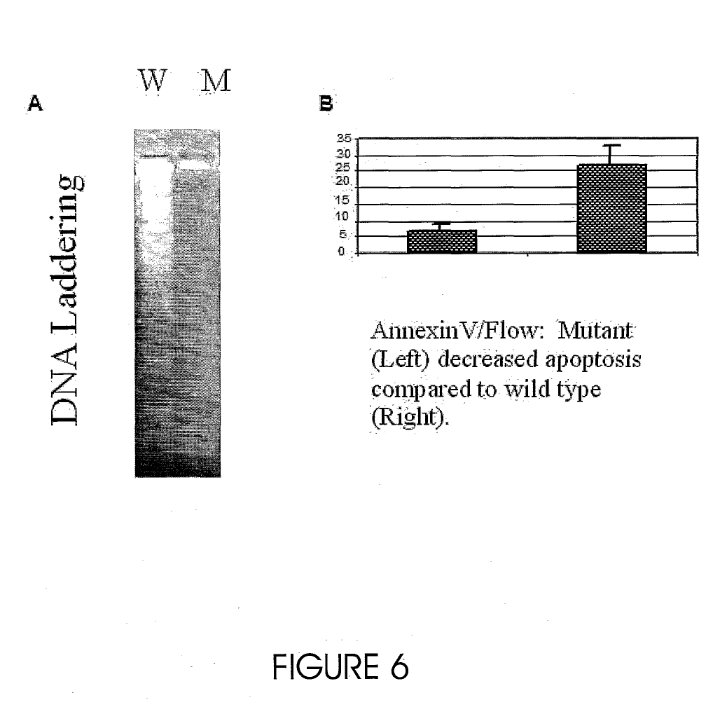

The 8993 mutation decreases apoptotic index and increases in vitro proliferation. The prostate cancer cell cybrids with the pathogenic np 8993G mutation were found to proliferate in vitro 50% faster than those with the wild type np 8993T variant (FIG. 5). Furthermore, the mutant lines had a 4-fold reduction in in vitro baseline apoptosis, as determined by annexin-V immuno-fluorescent flow cytometry. They also showed decreased DNA laddering, another marker of apoptosis (FIG. 6).

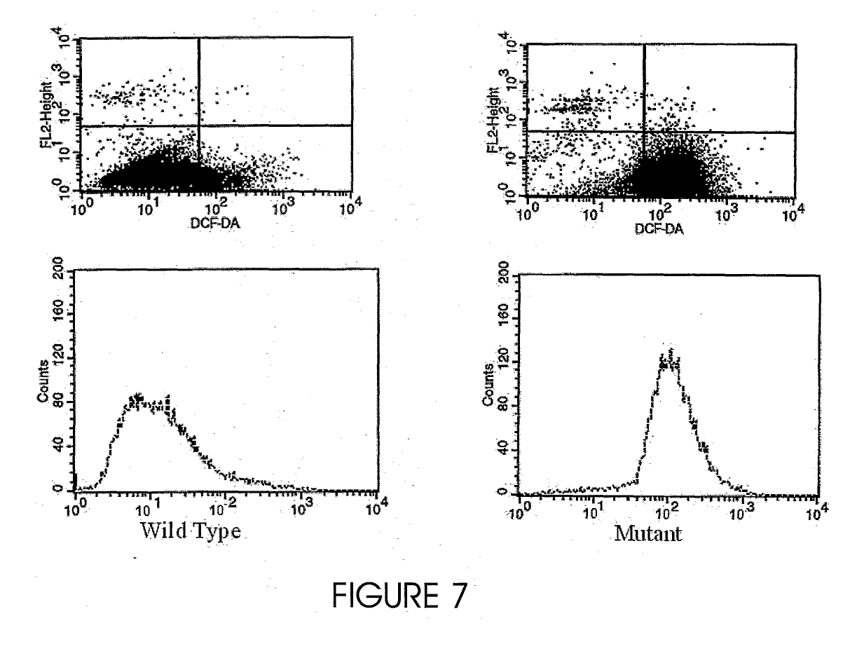

The mutant PC-3 cybrids had 10-fold higher total cellular ROS production as measured by flow cytometry monitoring dichlorodifluorescin (DCF) fluorescence (FIG. 7). Cells were grown for 3 days in 100 mm plates (70-90% confluence). Fresh media was added 12 hours before assay. Cells were trypsinized, washed with HBSS+5% FBS, and counted. In 1 mL HBSS+5% FBS, 2×10⁶ cells were incubated at room temperature in the dark for 30 minutes with a final concentration of 5 μM DCF-DA (2′,7′-dichlorofluorescin diacetate; Molecular Probes Inc. (Eugene, Oreg.)) and 10 μL propidium iodide (Sigma). Following incubation, samples were kept on ice until counting on a Becton Dickinson FACScan flow cytometer. Between 10,000 and 50,000 cells were counted and analyzed by CellQuest to compare mean values of DCF fluorescence intensity on PI negative cells. Samples were repeated in duplicate or triplicate. DCF-DA easily diffuses through the cell membrane where the actate groups are cleaved by alkaline hydrolysis trapping the polar DCFH within the cell. DCFH is oxidized by ROS to a highly fluorescent dye (DCF) (excitation 488 nm; emission 525 nm).

### Discussion

The current study shows that mtDNA mutations play an important role in the etiology of prostate cancer. Prostate cancers have a significantly increased frequency of functionally important COI mutations and the introduction into prostate cancer cells of an mtDNA mutation which inhibits OXPHOS and increases ROS production increased their tumor growth.

The COI mutations that we identified in prostate cancer fulfilled all of the criteria expected for mtDNA mutations that cause this disease. The COI mutations were significantly more frequent in prostate cancer patients than in no-cancer controls or in the general population. The COI mutations altered significantly more conserved amino acids, and they included both new heteroplasmic somatic and recurrent homoplasmic germline mutations. mtDNA COI mutations are a causal factor in the etiology of prostate cancer.

Germline COI mutations were also found to be an important risk factor for developing prostate cancer. COI missense mutations were common in the general population (7.8%), yet virtually absent (<2%) in cancer negative controls. Thus most men harboring COI missense mutations must move into the prostate cancer category by late middle age. The association between germline COI mtDNA mutations and prostate cancer risk might also explain why African American men are more prone to prostate cancer than European American men⁶⁹. Overall COI variants are relatively common in African mtDNA (17.4%, Table 2), in part due to certain African mtDNA lineages harboring ancient COI protein polymorphisms (e.g., the np T7389C and A7146G variants in African lineages L0 and LOL1)⁵⁴. Therefore, these ancient COI protein polymorphisms contribute to an increased predisposition to prostate cancer in African American men today.

Given that highly conserved COI mtDNA missense variants (CI=70%) are so common in the general population (7.8%), why aren't COI mutations more commonly found in neuromuscular disease? One possibility is that the human cell has the capacity to partially compensate for complex IV defects by changing the expression of the COX subunits. Mice lacking liver ANTs were found to selectively up-regulate Complex IV two fold⁷⁰. Hence, the biochemical effects of partial complex IV defects might be ameliorated by altered complex IV gene expression. This would be consistent with the observation that prostate cancers have increased levels of nDNA-encoded to mtDNA-encode complex IV subunits^{61,62}. Even so, the COI mutations would inhibit the ETC, and this could chronically increase mitochondrial ROS production and stimulate cell proliferation^{27,29,35}.

Since prostate cancer is the most common clinical consequence of COI mutations, this explains why COI mutations are so common in the general population. Prostate cancer kills middle aged or older males, but the mtDNA is exclusively maternally inherited. Hence, deleterious COI mutations that cause prostate cancer would have minimal effect on the genetic fitness of the mutant mtDNA.

Mutations in the mtDNA that inhibit the ETC and increase ROS production can act as both tumor promoters and tumor initiators. That mtDNA mutations which increase ROS production can be potent tumor promoters was demonstrated by our introduction of the pathogenic mtDNA ATP6 T8993G mutation^{58,68} into PC3 cells and showing a dramatic increased tumor growth rate in association with increased cellular ROS production. Moreover, the lack of tumor growth observed for the PC3(mtDNA T8993T) wild type cybrids might also support this conclusion, since it is well established that PC3 cells readily form tumors in Nude mice. Since the PC3 mtDNA was found to harbor a conserved ND5 np C13802T (T489M) mutation, it is possible that removal of this mutation reduced the tumorigenic potential of the PC3 cells.

That mtDNA mutations might also serve as tumor initiators was suggested by Tumor 18, which harbored both a germine ATP6 P136G and a somatic COI G16X mutation. Since the germine ATP6 mutation must have preceded that COI G16X mutation, it is possible that ROS generated as a result of the ATP6 P136G mutation could have damaged to the mtDNA and caused the COI G16X mutation.

These observations also indicate that mtDNA variants could have accounted for earlier somatic cell genetic observations that the cybrid transfer of chloramphenicol resistant (CAP^{R}) mtDNAs from a non-tumorigenic Chinese hamster cell line into a tumorigenic cell line suppressed tumorigenesis⁷¹, while the reciprocal transfer had no effect. Similarly, the transfer of CAP^{R} mtDNAs from the near euploid HT1080 cells into the aneuploid HeLa cells suppressed growth, but the reciprocal transfer caused no change⁷².

Our demonstration that partial defects in OXPHOS which increase ROS contribute to cancer now provides an explanation for the observation of Otto Warburg more than 70 years ago that solid tumors have a high rate of “aerobic-glycolysis”⁷³. Mutations that inhibit OXPHOS would not only make more ROS, they would oxidize less pyruvate and NADH. The pyruvate and NADH would be converted to lactate by lactate dehydrogenase resulting excessive lactate production during aerobic respiration, “aerobic-glycolysis,” a physiological state has been documented for cells harboring the ATP6 T8993G mutation⁷⁴. If mitochondrial ROS production is essential for solid tumor promotion, then “aerobic glycolysis” should be a common feature of solid tumors, which Warburg noted.

In conclusion, this study has revealed that mtDNA mutations are not only associated with a predisposition to neuromuscular disease, but also a predisposition to cancer. Therefore, we can now add cancer to the list of mitochondrial diseases.

TABLE 1
Prostate cancer-associated COI mutations.

| MUT | AA | Cl | HG | PROSTATE | BLOOD | CASE |
|---|---|---|---|---|---|---|
| C5911T | A3V | 13 | U | M | M | 18 |
| G5913A | D4N | 18 | K | M | M | 8 |
| A5935G | N11S | 100 | N | M | M* | 29 |
| G5949A | G16X | — | U | M/W | W | 18 |
| G5973A | A24T | 92 | H | M | M | 3 |
| G6081A | A60T | 97 | L2 | M | M | 31 |
| G6150A | V83I | 95 | L1 | M | M | 21 |
| T6124C | M74T | 95 | T | M/W | M/W | 19 |
| T6253C | M117T | 69 | H | M | M | 1, 2, 4 |
| G6261A | A120T | 97 | J, T, | M | M | 6, 7, 17, |
| | | | L1, N | | | 26, 27, 30 |
| G6267A | A122T | 92 | L1 | M | M | 24 |
| G6285A | V128I | 100 | H | M | M | 16 |
| C6340T | T146I | 79 | H, N | M | M* | 5, 23 |
| G6480A | V193I | 87 | 1 | M | M | 14 |
| A6663G | I254V | 95 | O, L2 | M | M | 12, 13, 20, |
| | | | | | | 22, 25 |
| G6924T | A341S | 100 | K | M | W | |
| G7041A | V380I | 100 | T | M | M | 9 |
| T7080C | F393L | 97 | U | M | M* | 10 |
| A7083G | I395V | 33 | H | M | M | 15 |
| A7158G | I419V | 21 | N | M | M* | 28 |
| A7305C | M468L | 90 | U | M | M | 11 |

M = Mutant,
W = Wild Type.
*Four cases had no blood available.
DNA sequencing of a separate organ (seminal vesicle) in these 4 cases confirmed these as germ line mutations. 7389 and 7146, defining L0 and L0L1 respectively, were not considered.

TABLE 2
Frequency of COI mutations in prostate cancer patients, controls, and the
general population.

| | N | COI MUTANT | FREQUENCY (%) | P (FET) |
|---|---|---|---|---|
| CANCER | 260 | 31 | 11.0^{1,b} | |
| EA | 180 | 19 | 10.6^{c,d} | |
| AA | 80 | 12 | 15.0 | |
| NO-CANCER | 54 | 1 | 1.9^{a} | 0023 |
| EA | 46 | 0 | 0^{c} | 0.016 |
| AA | 8 | 1 | 12.5 | 0.674 |
| POPULATION | 1338 | 104 | 7.8 | |
| EA + AA | 1019 | 79 | 7.8^{b} | 0.015 |
| EA | 898 | 58 | 6.5^{d} | 0.025 |
| AA | 121 | 21 | 17.4 | 0.432 |
| NON (EA + AA) | 319 | 25 | 7.8 | |

Frequencies with the same superscript were compared and the P value represented at the right column. The 1338 sequences labeled “population” have no clinical information but serve as a population frequency control. Note that only amino acid altering mutations are included and that the ancient African-defining missense mutations at np7389 is excluded. The frequency of this mutation appears in a separate data set described below. EA=European Americans, AA=African Americans. FET is Fisher's exact test.

### Haplotype Determination

We analyzed 121 consecutive Caucasian patients that had undergone radical nephrectomy in the 1990's for renal cancer (age range 17 to 88, average age 58.40±13.58) and 221 consecutive prostate cancer patients accrued between 1997 and 2001 (age range 40 to 81, average age 56.71±8.49). Data gathering and analysis were done in compliance with federal and institutional regulations (Health Insurance Portability and Accountability Act and Emory University's Institutional Review Board).

Because there are slight differences in mitochondrial haplogroups in Caucasians from different regions of the United States (due to immigration patterns) we have chosen a control group (246 patients, age range 10 months to 69 years, average age 34.21±16.55) from the same region as the cancer patients, making the differences in haplogroup distribution between cases and controls due only to disease status and not regional variation or ethnic origin.

Source of DNA. Total genomic DNA was prepared from fresh frozen tissue procured prospectively as part of ongoing tissue banking for the 121 renal cancer patients and 157 of the prostate cancer patients. An additional 64 prostate cancer tissue DNAs were prepared from paraffin-embedded tissues. The control DNAs were prepared from frozen tissues obtained from 246 organ donors at Emory University Hospital. Because mitochondrial haplogroup assignment depends upon the identification of a limited set of precisely defined polymorphisms equally present in every cell in the body, no attempt was made to differentiate cancerous from normal tissue.

Determination of mtDNA Haplogroup. North American individuals of European origin belong to one of nine mitochrondrial haplogroups (H, I, J, K, T, U, V, W or X)^{75,76}. Primers were designed in order to amplify the region of mitochondrial DNA that defines each haplogroup (Table 1). Standard PCR amplification conditions were used followed by digests that define European haplogroups. A haplogroup assignment was confirmed when a polymorphic mutation was present in the mitochondrial DNA as listed in Table 1. The frequency of individual haplogroups of cancer patients was then statistically compared to controls using a Fisher's exact test (two-tail). Given the nine haplogroups considered, there were nine tests performed in the comparison between each group of cancer patients and controls. In order to consider the effects of multiple testing, the alpha level was adjusted downward by applying a conservative Bonferroni correction. Odds ratios (OR) were also calculated for the cancer patients in each haplogroup. Due to the relatively low prevalence of prostate and renal cancers (prostate cancer is one of the most commonly diagnosed malignancies in men, but its incidence is relatively low when compared to nonmalignant diseases), OR estimates are valid estimates of the cancer relative risk for the different haplogroups (Table 3).

### Results and Discussion

206 of the 221 prostate cancer patients and 110 of the 121 renal cancer patients fell into the 9 mitochondrial haplogroups analyzed in this study (H, I, J, K, T, U, V, W, and X). All of the remaining cancer patients were grouped as “other,” a group that is expected to contain Caucasian North Americans who may not be of European heritage (e.g. haplotype N1b). Following the assignment of haplogroup, the prevalence of each haplogroup was determined for each cancer group. There were no significant differences between cancer groups and the regional control distributions for haplogroups H, I, J, K, V, W, and X. Haplogroup U was significantly over represented in both prostate and renal cancer patients while haplogroup T was underrepresented in renal cancer patients (Table 3), an association that just misses statistical significance. In order to assess the effect of the “other” group on these results, the statistical analyses were repeated excluding this group. The results obtained in terms of significance and alpha level were essentially the same and remained significant (data not shown).

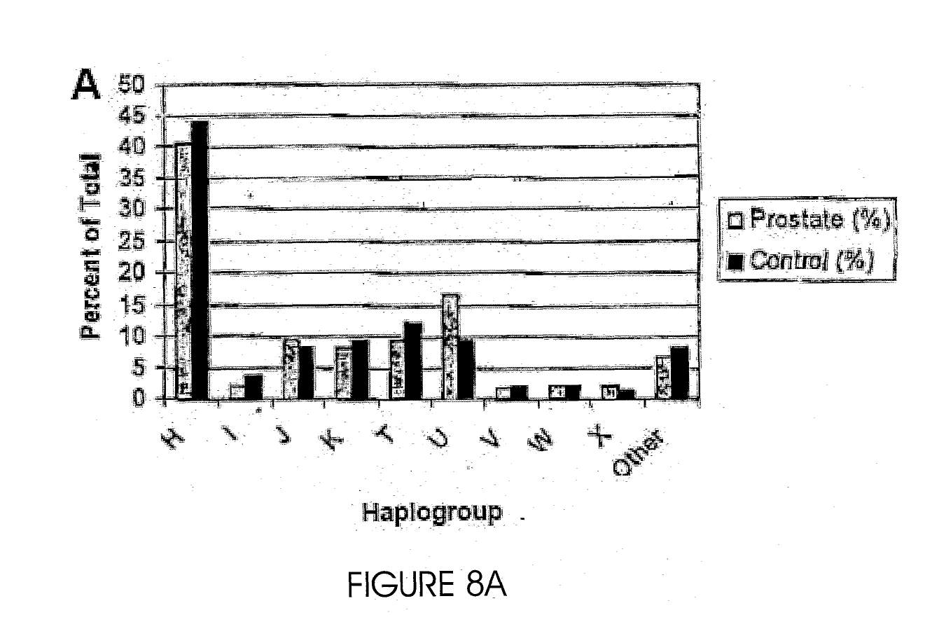

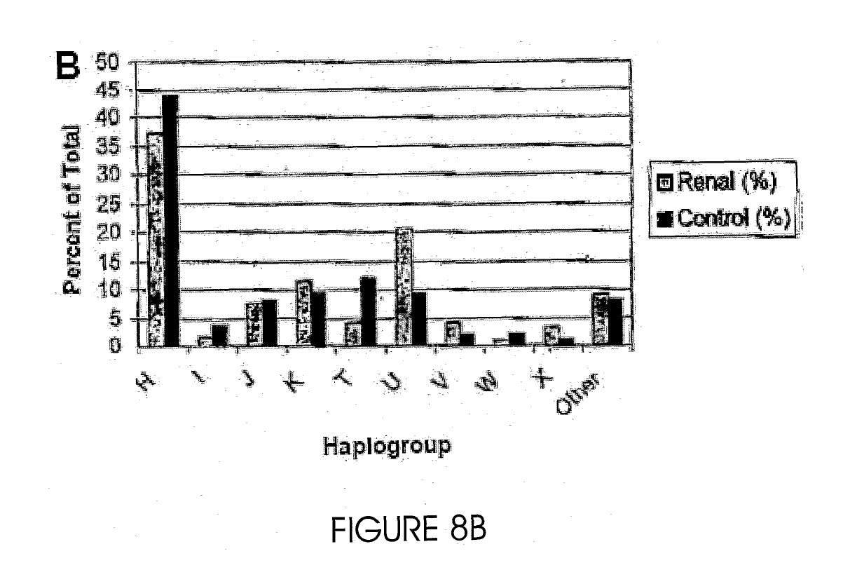

When the haplogroup distribution of prostate cancer patients was compared to the control group, a significantly greater percentage of prostate cancer patients were in the mitochondrial haplogroup U. 16.74% (37/221) of the prostate cancer population compared to 9.35% (23/246) of the regional control population were members of the U haplogroup (Fisher's exact p=0.019). The OR for developing prostate cancer for individuals in mitochondrial haplogroup U is 1.95 which suggests that the U haplogroup is a prostate cancer risk factor (Table 3, FIG. 8a).

When comparing the haplogroup distribution for renal cancer patients to the control group, there is again a significantly greater percentage of renal cancer patients in the mitochondrial haplogroup U: 20.66% (25/121) compared to 9.35% (23/246) of the control population were members of this haplogroup (Fisher's exact p=0.005). The p-value for the increased prevalence of the U haplogroup among renal cancer patients also remained statistically significant at the 0.05 level after a conservative Bonferroni adjustment. The OR for developing renal cancer for individuals in mitochondrial haplogroup U is 2.52 which suggests that the U haplogroup is a renal cancer risk factor (Table 3, FIG. 8b). A significantly smaller percentage of renal cancer patients in the T haplogroup designation was also noted: 4.13% (5/121) of the renal cancer population compared to 12.20% (30/246) of the control population were members of the T haplogroup (Fisher's exact p=0.013). When a conservative Bonferroni adjustment was applied, the p-value for the decreased prevalence of the T haplogroup among renal cancer patients did not remain statistically significant. However, the significance level obtained with the Fisher's exact test combined with the low OR value for individuals in mitochondrial haplogroup T (OR=0.31) make this haplogroup a potential protective factor against renal cancer (Table 3, FIG. 8b).

The disease status of the control group is not known. Because some of the controls are likely to have (or subsequently develop) prostate or renal cancer, the differences we found are the minimal possible differences, and the presence of unidentified cancer patients in the control group would only enhance the statistical significance of the distributional differences we found if patients could be culled from the control group. The mean age of the controls (34 y/o) is less than that of the cancer cases (56-58 y/o). The effect of this age difference is minimal as their disease status was not determined and controls were chosen because they represent the regional population's inherited mtDNA haplogroup distribution, which does not change with age or disease state. In this study, we have documented that the distributions of mitochondrial haplogroups in Caucasian North American renal and prostate cancer patients are different from a regional control group of North American Caucasians. Overrepresentation of the U haplogroup was seen for the renal cancer group at highly significant levels, an association that remained significant even after a very conservative adjustment for the possible effect of multiple comparisons. Underrepresentation of haplogroup T in renal cancer patients and overrepresentation of haplogroup U in prostate cancer patients was also observed, though not significant following a conservative statistical (Bonferonni) analysis. These findings confirm that mitochondrial composition serves as either a predisposing or protective factor for the subsequent development of these two common adult solid tumors.

One consequence of having a single mitochondrial haplogroup that is predisposed to the development of both prostate and renal cancer is that there should be an increased incidence of renal cancer in prostate cancer patients and an increased rate of prostate cancer in renal cancer patients. A review of the literature confirms that both are true.

A population based study of all men diagnosed with prostate cancer (N=3675) in the metropolitan Atlanta area between 1975 and 1982 clearly documented an increased rate of renal cancer in the study group compared to the local general population⁷⁷. A similar study was performed using the New South Wales (Australia) central cancer registry for the period 1972-1991 to determine the risk of second malignancies following initial renal cancer or an initial prostate cancer. There was a reciprocal relationship of increased risk of prostate cancer (RR=1.7) following an initial renal cancer and an increased risk of renal cancer following an initial prostate cancer (RR=1.2)⁷⁸. A small cohort study of 164 men with prostate cancer in Connecticut revealed an increased rate of subsequent renal cancers compared to the general incidence in that state⁷⁹. A cohort of 763 patients with renal cancer treated surgically in New York showed and increased risk of developing prostate cancer after an initial diagnosis of renal cancer (papillary histology)⁸⁰.

Two studies of familial cancers in Scandinavian countries are particularly interesting. In the first study, 62 Swedish families with hereditary prostate carcinoma (HPC) were studied and the overall cancer risk was observed amongst 1364 first-degree relatives⁸¹. The standardized incidence ratio (SIR) was defined as the ratio between the observed and the expected number of cases. There was a significant increased risk for renal cancer, but only amongst female first-degree relatives (SIR 4.60, 95% CI 1.84-9.48). This was the highest ratio of all cancers studied. The second study analyzed the relative risk of all cancers amongst relatives of 371 Icelandic men with prostate cancer⁸². They found that the risk of kidney cancer was higher in first- and second-degree female relatives with a RR of 2.5 and 2.67 respectively. The risk of kidney cancer was not statistically significantly greater in male relatives. The increased risk of cancer for female relatives only is consistent with a mitochondrial predisposition factor.

We have shown that inheriting mitochondrial haplogroup U confers increased risk for prostate and renal cancer in North American Caucasians. For the first time, mitochondrial haplogroups have been shown to play a role as predisposing factors in human cancer. We conclude that the mitochondrial haplogroup U predisposes to renal cancer, while haplogroup T may be protective for renal cancer and haplogroup U may predispose to prostate cancer.

This invention has been exemplified using specific mutations, detection methods, and other specific reagents and methods. However, as will be appreciated by one skilled in the art, equivalents of such specifics can be substituted therefor.

TABLE 3
Mitochondrial Haplogroup Frequencies in Cases and Controls

| | Polymorphic | Primer | Control | | Prostate | | |
|---|---|---|---|---|---|---|---|
| Haplogroup | sites^{a} | coordinates^{b} | n | % | n | % | OR |
| H | −7025Alul | 6890-6909, | 108 | 43.90 | 90 | 40.72 | 0.88 |
| | | 7131-7115 | | | | | |
| I | −1715Ddel | 1615-1643, | 9 | 3.66 | 5 | 2.26 | 0.61 |
| | | 1894-1874 | | | | | |
| | +8249Avall | 8188-8207, | | | | | |
| | | 8366-8345 | | | | | |
| | +10028Alul | 9911-9932, | | | | | |
| | | 10107-10088 | | | | | |
| J | −16065Hinfl | 15838-15857, | 20 | 8.13 | 21 | 9.50 | 1.19 |
| | | 16261-16242 | | | | | |
| K | −9052Haell | 8829-8845, | 23 | 9.35 | 18 | 8.15 | 0.86 |
| | | 9184-9163 | | | | | |
| | +12308Hinfl | 12104-12124, | | | | | |
| | | 12338-12309 | | | | | |
| T | +13366BamHl | 13172-13190, | 30 | 12.20 | 21 | 9.50 | 0.76 |
| | | 13403-13384 | | | | | |
| | +15606Alul | 15409-15428, | | | | | |
| | | 15701-15682 | | | | | |
| U | +12308Hinfl | 12104-12124, | 23 | 9.35 | 37c | 16.74 | 1.95 |
| | | 12338-12309 | | | | | |
| V | −4577Nlalll | 4500-4519, | 5 | 2.03 | 4 | 1.81 | 0.89 |
| | | 4680-4661 | | | | | |
| W | +8249Avall | 8188-8207, | 5 | 2.03 | 5 | 2.26 | 1.12 |
| | | 8366-8345 | | | | | |
| | −8994Haelll | 8829-8845, | | | | | |
| | | 9184-9163 | | | | | |
| X | −1715Ddel | 1615-1643, | 3 | 1.22 | 5 | 2.26 | 1.87 |
| | | 1894-1874 | | | | | |
| Other | | | 20 | 8.13 | 15 | 6.79 | 0.82 |
| Total | | | 246 | | 221 | | |

### REFERENCES

1. American Cancer Society. Cancer facts and figures 2005. Atlanta: American Cancer Society; 2005.
2. Oda, T., Takahashi, A., Miyao, N., Yanase, M., Masumori, N., ltoh, N., et al. Cell proliferation, apoptosis, angiogenesis and growth rate of incidentally found renal cell carcinoma. Int. J. Urol., 10:13-18, 2003.
3. Catz, S. D., and Johnson, J. L. BCL-2 in prostate cancer: a minireview. Apoptosis, 8:29-37, 2003.
4. Horton, T. M., Petros, J. A., Heddi, A., Shoffner, J., Kaufman, A. E., Graham, S. D., Jr., Gramlich, T. & Wallace, D. C. (1996) Genes, Chromosomes and Cancer 15, 95-101.
5. Chen, J. Z., Gokden, N., Greene, G. F., Mukunyadzi, P. & Kadlubar, F. F. (2002) Cancer Research 62, 6470-6474.
6. Chinnery, P. F., Samuels, D. C., Elson, J. & Turnbull, D. M. (2002) Lancet 360, 1323-1325.
7. Jeronimo, C., Nomoto, S., Caballero, O. L., Usadel, H., Henrique, R., Varzim, G., Oliveira, J., Lopes, C., Fliss, M. S. & Sidransky, D. (2001) Oncogene 20, 5195-5198.
8. Jessie, B. C., Sun, C. Q., Irons, H. R., Marshall, F. F., Wallace, D. C., Petros, J. A., Accumulation of mitochondrial DNA deletions in the malignant prostate of patients of different ages. Experimental Gerontology. 37(1):169-74, 2001 December.
9. Copeland, W. C., Wachsman, J. T., Johnson, F. M. & Penta, J. S. (2002) Cancer Investigation 20, 557-569.
10. Liang, B. C. Evidence for association of mitochondrial DNA sequence amplification and nuclear localization in human low-grade gliomas. Mutation Res 354:27-33, 1996.
11. Alonso, A., Martin, P., Albarran, C., Aguilera, B., Garcia, O., Guzman, A., Oliva, H., Sancho, M. Detection of somatic mutations in the mitochondrial DNA control region of colorectal and gastric tumors by heteroduplex and single-strand conformation analysis. Electrophoresis 18:682-685, 1997.
12. Polyak, K., Li, Y., Lengauer, C., Willson, J. K. V., Markowitz, S. D., Trush, M. A., Kinzler, K. W., Vogelstein, B. Somatic mutations of the mitochondrial genome in human colorectal tumors. Nature Genet 20:291-293, 1998.
13. Bianchi, N. O., Bianchi, M. S., Richard, S. M. Mitochondrial genome instability in human cancers. Mutation Research. 488(1):9-23, 2001 Mar.
14. Kirches, E., Michael, M., Woy, C., Schneider, T., Warich-Kirches, M., Schneider-Stock, R., Winkler, K., Wittig, H., Dietzmann, K. Loss of heteroplasmy in the displacement loop of brain mitochondrial DNA in astrocytic tumors. Genes, Chromosomes & Cancer. 26:80-3, 1999.
15. Fliss, M. S., Usadel, H., Caballero, O. L., Wu, L., Buta, M. R., Eleff, S. M., Jen, J., Sidransky, D. Facile detection of mitochondrial DNA mutations in tumors and bodily fluids. Science 287:2017-2019, 2000.
16. Warburg, O. On the origin of cancer cells. Science, 123:309-314, 1956.
17. Haga, N., Fujita, N., and Tsuruo, T. Mitochondrial aggregation precedes cytochrome c release from mitochondria during apoptosis. Oncogene, 22:5579-5585, 2003.
18. Gao, N., Ding, M., Zheng, J. Z., Zhang, Z., Leonard, S. S., Liu, K. J., et al. Vanadate-induced expression of hypoxia-inducible factor 1 alpha and vascular endothelial growth factor through phosphatidylinositol 3-kinase/Akt pathway and reactive oxygen species. J. Biol. Chem., 277:31963-31971, 2002.
19. Astuti, D., Latif, F., Dallol, A., Dahia, P. L., Douglas, F., George, E., Skoldberg, F., Husebye, E. S., Eng, C. & Maher, E. R. (2001) American Journal of Human Genetics 69, 49-54.
20. Baysal, B. E., Ferrell, R. E., Willett-Brozick, J. E., Lawrence, E. C., Myssiorek, D., Bosch, A., van der Mey, A., Taschner, P. E., Rubinstein, W. S., Myers, E. N. et al. (2000) Science 287, 848-851.
21. Niemann, S. & Muller, U. (2000) Nature Genetics 26, 268-270.
22. Vanharanta, S., Buchta, M., McWhinney, S. R., Virta, S. K., Peczkowska, M., Morrison, C. D., Lehtonen, R., Januszewicz, A., Jarvinen, H., Juhola, M. et al. (2004) American Journal of Human Genetics 74, 153-159.
23. Bourgeron, T., Rustin, P., Chretien, D., Birch-Machin, M., Bourgeois, M., Viegas-Pequignot, E., Munnich, A. & Rotig, A. (1995) Nature Genetics 11, 144-149.
24. Arbiser, J. L. (2004) Seminars in Cancer Biology 14, 81-91.
25. Arbiser, J. L., Petros, J., Klafter, R., Govindajaran, B., McLaughlin, E. R., Brown, L. F., Cohen, C., Moses, M., Kilroy, S., Arnold, R. S., Lambeth, J. D. Reactive oxygen generated by Nox1 triggers the angiogenic switch. Proceedings of the National Academy of Sciences USA 99(2):715-720, 2002.
26. Simmons, R. A., Suponitsky-Kroyter, I., Selak, M. A. Progressive accumulation of mitochondrial DNA mutations and decline in mitochondrial function lead to beta-cell failure. Journal of Biological Chemistry. 280(31):28785-91, 2005 Aug. 5.
27. Wallace, D. C. & Lott, M. T. (2002) in Emery and Rimoin's Principles and Practice of Medical Genetics, eds. Rimoin, D. L., Connor, J. M., Pyeritz, R. E. & Korf, B. R. (Churchill Livingstone, London), pp. 299-409.
28. Evans, A. R., Limp-Foster, M. & Kelley, M. R. (2000) Mutation Research 461, 83-108.
29. McCord, J. M. (2000) American Journal of Medical Genetics 108, 652-659.
30. Lee, S. R., Kwon, K. S., Kim, S. R. & Rhee, S. G. (1998) Journal of Biological Chemistry 273, 15366-15372.
31. Haddad, J. J. (2002) European Cytokine Network 13, 250-260.
32. Michiels, C., Minet, E., Mottet, D. & Raes, M. (2002) Free Radical Biology and Medicine 33, 1231-1242.
33. Baker, A. M., Oberley, L. W. & Cohen, M. B. (1997) Prostate 32, 229-233.
34. Bostwick, D. G., Alexander, E. E., Singh, R., Shan, A., Qian, J., Santella, R. M., Oberley, L. W., Yan, T., Zhong, W., Jiang, X. et al. (2000) Cancer 89, 123-134.
35. Xu, Y., Krishnan, A., Wan, X. S., Majima, H., Yeh, C. C., Ludewig, G., Kasarskis, E. J. & St. Clair, D. K. (1999) Oncogene 18, 93-102.
36. Esposito, L. A., Melov, S., Panov, A., Cottrell, B. A. & Wallace, D. C. (1999) Proceedings of the National Academy of Sciences of the United States of America 96, 4820-4825.
37. Graham, B., Waymire, K., Cottrell, B., Trounce, I. A., MacGregor, G. R. & Wallace, D. C. (1997) Nature Genetics 16, 226-234.
38. Taylor, R. W., Turnbull, D. M. Mitochondrial DNA mutations in human disease. Nature Reviews Genetics. 6(5):389-402, 2005 May.
39. Lawen, A., Ly, J. D., Lane, D. J. R., Zarschler, K., Messina, A., Pinto, V. D. Voltage-dependent anion-selective channel 1 (VDAC1)—a mitochondrial protein, rediscovered as a novel enzyme in the plasma membrane. International Journal of Biochemistry & Cell Biology 37:277-282, 2005.
40. Dorward, A., Sweet, S., Moorehead, R., Singh, G. Mitochondrial contributions to cancer cell physiology: Redox balance, cell cycle and drug resistance. Journal of Bioenergetics & Biomembranes 29(4):385-392, 1997 August.
41. Ruiz-Pesini, E., Mishmar, D., Brandon, M., Procaccio, V., Wallace, D. C. Effects of purifying and adaptive selection on regional variation in human mtDNA. Science 303:223-226, 2004.
42. Ruiz-Pesini, E., Lapena, A. C., Diez-Sanchez, C., Perez-Martos, A., Montoya, J., Alvarez, E., Diaz, M., Urries, A., Montoro, L., Lopez-Perez, M. J., Enriquez, J. A. Human mtDNA haplogroups associated with high or reduced spermatozoa motility. American Journal of Human Genetics. 67(3):682-96, 2000 Sep.
43. Kalman, B., Lublin, F. D., Alder, H. Characterization of the mitochondrial DNA in patients with multiple sclerosis. Journal of the Neurological Sciences. 140(1-2):75-84, 1996 Sep. 1.
44. Schroder, F. H. Detection of prostate cancer: the impact of the European Randomized Study of Screening for Prostate Cancer (ERSPC). Canadian Journal of Urology. 12 Suppl 1:2-6; 2005 Feb.
45. Petros, J. A., Baumann, A. K., Ruiz-Pesini, E., Amin, M. B., Sun, C. Q., Hall, J., Lim, S., Issa, M. M., Flanders, W. D., Hosseini, S. H., Marshall, F. F., Wallace, D. C. mtDNA mutations increase tumorigenicity in prostate cancer. Proceedings of the National Academy of Sciences of the United States of America. 102(3):719-24, 2005 Jan. 18.
46. Valeri, A., Briollais, L., Azzouzi, R., Fournier, G., Mangin, P., Berthon, P., Cussenot, O., Demenais, F. Segregation analysis of prostate cancerin France: evidence for autosomal dominant inheritance and residual brother-brother dependence. Annals of Human Genetics. 67(Pt 2):125-37, 2003 Mar.
47. Bochum, S., Paiss, T., Vogel, W., Herkommer, K., Hautmann, R., Haeussler, J. Confirmation of the prostate cancer susceptibility locus HPCX in a set of 104 German prostate cancer families. Prostate. 52(1):12-9, 2002 Jun. 1.
48. Ishii, N., Fujii, M., Hartman, P. S., Tsuda, M., Yasuda, K., Senoo-Matsuda, N., Yanase, S., Ayusawa, D. & Suzuki, K. (1998) Nature 394, 694-697.
49. Senoo-Matsuda, N., Yasuda, K., Tsuda, M., Ohkubo, T., Yoshimura, S., Nakazawa, H., Hartman, P. S. & Ishii, N. (2001) Journal of Biological Chemistry 276, 41553-41558.
50. Wallace, D. C., Lott, M. T., Brown, M. D., and Kerstann, K. Mitochondria and Neuro-opthalmologic Diseases. In: C. R. Scriver, A. L. Beaudet, W. S. Sly, D. Valle, B. Childs, and K. W. Kinzler (eds.), The Metabolic & Molecular Bases of Inherited Disease, Eighth Edition. pp. 2425-2491. New York: McGraw-Hill, 2001.
51. Kalman, B., Li, S., Chatterjee, D., O'Connor, J., Voehl, M. R., Brown, M. D., and Alder H. Large scale screening of the mitochondrial DNA reveals no pathogenic mutations but a haplotype associated with multiple sclerosis in Caucasians. Acta Neurol. Scand., 99:16-25, 1999.
52. Brown, M. D., Sun, F., and Wallace, D. C. Clustering of Caucasian Leber Hereditary Optic Neuropathy Patients Containing the 11778 or 14484 Mutations on an mtDNA Lineage. Am. J. Hum. Genet., 60:381-387, 1997.
53. Shoffner, J. M., Brown, M. D., Torroni, A., Lott, M. T., Cabell, M. F., Mirra, S. S., et al. Mitochondrial DNA Variants Observed in Alzheimer Disease and Parkinson Disease Patients. Genomics, 17:171-184, 1993.
54. Ruiz-Pesini, E., Mishmar, D., Brandon, M., Procaccio, V. & Wallace, D. C. (2004) Science 303, 223-226.
55. Mishmar, D., Ruiz-Pesini, E., Brandon, M. & Wallace, D. C. (2004) Human Mutation 23, 125-133.
56. Goldsworthy, S. M., Stockton, P. S., Trempus, C. S., Foley, J. F. & Maronpot, R. R. (1999) Molecular Carcinogenesis 25, 86-91.
57. Trounce, I. A., Kim, Y. L., Jun, A. S. & Wallace, D. C. (1996) Methods in Enzymology 264, 484-509.
58. Trounce, I., Neill, S. & Wallace, D. C. (1994) Proceedings of the National Academy of Sciences of the United States of America 91, 8334-8338.
59. Nakamura, Y., Leppert, M., O'Connell, P., Wolff, R., Holm, T., Culver, M., Martin, C., Fujimoto, E., Hoff, M., Kumlin, E. et al. (1987) Science 235, 1616-1622.
60. Bassoe, C. F., Li, N., Ragheb, K., Lawler, G., Sturgis, J., Robinson, J. P. Investigations of phagosomes, mitochondria, and acidic granules in human neutrophils using fluorescent probes. Cytometry Part B, Clinical Cytometry. 51(1):21-9, 2003 Jan.
61. Herrmann, P. C., Gillespie, J. W., Charboneau, L., Bichsel, V. E., Paweletz, C. P., Calvert, V. S., Kohn, E. C., Emmert-Buck, M. R., Liotta, L. A. & Petricoin, E. F., 3^{rd} (2003) Proteomics 3, 1801-1810.
62. Krieg, R. C., Knuechel, R., Schiffmann, E., Liotta, L. A., Petricoin, E. F., 3^{rd} & Hermann, P. C. (2004) Proteomics 4, 2789-2795.
63. Taylor, R. W., Barron, M. J., Borthwick, G. M., Gospel, A., Chinnery, P. F., Samuels, D. C., Taylor, G. A., Plusa, S. M., Needham, S. J., Greaves, L. C. et al. (2003) Journal of Clinical Investigation 112, 1351-1360.
64. Broker, S., Meunier, B., Rich, P., Gattermann, N. & Hofhaus, G. (1998) European Journal of Biochemistry 258, 132-138.
65. Gattermann, N., Retzlaff, S., Wang, Y. L., Hofhaus, G., Heinisch, J., Aul, C. & Schneider, W. (1997) Blood 90, 4961-4972.
66. Mishmar, D., Ruiz-Pesini, E. E., Golik, P., Macaulay, V., Clark, A. G., Hosseini, S., Brandon, M., Easley, K., Chen, E., Brown, M. D. et al. (2003) Proceedings of the National Academy of Sciences of the United States of America 100, 171-176.
67. Holt, I. J., Harding, A. E., Petty, R. K. & Morgan-Hughes, J. A. (1990) American Journal of Human Genetics 46, 428-433.
68. Mattiazzi, M., Vijayvergiya, C., Gajewski, C. D., DeVivo, D. C., Lenaz, G., Wiedmann, M. & Manfredi, G. (2004) Human Molecular Genetics 13, 869-879.
69. Sakr, W. A. (2004) Modern Pathology: an Official Journal of the United States and Canadian Academy of Pathology, Inc., 1-10.
70. Kokoszka, J. E., Waymire, K. G., Levy, S. E., Sligh, J. E., Cai, J., Jones, D. P., MacGregor, G. R. & Wallace, D. C. (2004) Nature 427, 461-465.
71. Howell, A. N. & Sager, R. (1978) Proceedings of the National Academy of Sciences of the United States of America 75, 2358-2362.
72. Wallace, D. C. (1981) Molecular and Cellular Biology 1, 697-710.
73. Warburg, O. (1931) The Metabolism of Tumors (R. R. Smith, New York).
74. Pallotti, F., Baracca, A., Hernandez-Rosa, E., Walker, W. F., Solaini, G., Lenaz, G., Melzi D'Eril, G. V., DiMauro, S., Schon, E. A. & Davidson, M. M. (2004) Biochemical Journal Epub ahead of print.
75. Torroni, A., Huoponen, K., Francalacci, P., Petrozzi, M., Morelli, L., Scozzari, R., et al. Classification of European mtDNAs from an analysis of three European populations. Genetics, 144:1835-1850, 1996.
76. Torroni, A., Lott, M. T., Cabell, M. F., Chen, Y., Layergne, L., and Wallace, D. C. mtDNA and the Origin of Caucasians: Identification of Ancient Caucasian-specific Haplogroups, One of Which is Prone to a Recurrent Somatic Duplication in the D-Loop Region. Am. J. Hum. Genet., 55:760-776, 1994
77. Greenberg R. S., Rustin, E. D., Clark, W. S. Risk of Genitourinary Malignancies After Cancer of the Prostate. Cancer 61:396-401, 1988.
78. McCredie, M., Macfarlane, G. L., Stewart, J., Coates, M. Second Primary Cancers Following Cancers of the Kidney and Prostate in New South Wales (Australia), 1972-91. Cancer Causes & Control. 7:337-344, 1996.
79. Johnstone, P. A., Powell, C. R., Riffenburgh, R., Rohde, D. C., Kane, C. J. Second Primary Malignancies in T1-3N0 Prostate Cancer Patients Treated with Radiation Therapy with 10-year Followup. J. Urol. 159:946-949, 1998.
80. Rabbani, F., Reuter, V. E., Katz, J., Russo P. Second Primary Malignancies Associated with Renal Cell Carcinoma: Influence of Histologic Type. Urology 56:399-403, 2000.
81. Gronberg, H., Bergh, A., Damber, J. E., Emanuelsson, M. Cancer Riskin Families with Hereditary Prostate Carcinoma. Cancer, 89:1315-1321, 2000.
82. Eldon, B. J., Jonsson, E., Tomasson, J., Tryggvadottir, L., Tulinius H. Familial Risk of Prostate Cancer in Iceland. BJU International 92:915-919, 2003.

1. A method for identifying a subject likely to have, or at risk of developing a disease correlated with increased reactive oxygen species (ROS), said method comprising identifying in said subject a missense mutation in a nucleic acid of Complex III, IV, and/or V of the OXPHOS system.

2. The method of claim 1 wherein said disease is cancer.

3. The method of claim 1 wherein mutation is a missense mutation in the cytochrome c oxidase I (COI) gene.

4. The method of claim 2 wherein said subject does not suffer from a neuromuscular disease.

5. The method of claim 2 wherein said cancer is selected from the group consisting of colon cancer, lung cancer, kidney (renal) cancer, breast cancer, and prostate cancer.

6. The method of claim 2 wherein said cancer is prostate cancer.

7. The method of claim 2 wherein said cancer is kidney cancer.

8. The method of claim 1 wherein said mutation is selected from the group consisting of C5911T, G5913A, A5935G, G5949A, G5973A, G6081A, G6150A, T6124C, T6253C, G6261A, G6267A, G6285A, C6340T, G6480A, A6663G, G6924T, G7041A, T7080C, A7083G, A7158G, A7305C, A14769G, C8932T, and T7389C.

9. The method of claim 1 wherein said mutation is selected from the group consisting of G5949A, A14769G, and C8932T.

10. The method of claim 8 wherein at least two of said mutations are identified.

11. The method of claim 1 wherein said mutation has the effect of inhibiting OXPHOS and increasing ROS.

12. The method of claim 1 wherein said mutation is identified in a peripheral blood sample from said subject.

13. The method of claim 1 wherein said subject is a human.

14. The method of claim 1 also comprising counseling said subject relative to appropriate follow-up testing and treatment.

15. A method for detecting in a subject a condition selected from the group consisting of: likelihood of having cancer, being at risk of developing cancer, and likelihood of passing a predisposition to prostate cancer to progeny, comprising identifying in non-tumor tissue of said subject a missense mutation in a complex III, IV and/or V gene of the mitochondrial OXPHOS system in non-tumor tissue of said subject.

16. The method of claim 15 wherein said cancer is prostate cancer.

17. The method of claim 15 wherein said mutation is a mitochondrial mutation.

18. The method of claim 17 wherein said tissue is lymphocytes.

19. The method of claim 15 wherein said condition is being likely to develop prostate cancer and said mutation is homoplasmic or heteroplasmic in non-tumor tissue of said subject.

20. The method of claim 15 wherein said condition is a likelihood of passing a predisposition to prostate cancer to progeny, and said mutation is homoplasmic in non-tumor tissue of said subject.

21. The method of claim 16 wherein said subject is a human male, and said method also comprises further testing said subject for the presence of prostate cancer.

22. The method of claim 15 wherein said mutation is in a complex IV gene.

23. The method of claim 15 wherein said mutation is selected from the group consisting of C5911T, G5913A, A5935G, G5973A, G6081A, G6150A, T6124C, T6253C, G6261A, G6267A, G6285A, C6340T, G6480A, A6663G, G7041A, T7080C, A7083G, A7158G, A7305C, and T7389C.

24. The method of claim 23 comprising detecting at least two of said mutations.

25. A method for detecting the presence of a heritable predisposition to cancer in a subject comprising detecting in a non-tumor tissue of said subject a homoplasmic missense mutation in a gene of the OXPHOS system.

26. The method of claim 25 wherein said cancer is prostate cancer.

27. A nucleic acid array consisting essentially of probes made of normal sequences from the OXPHOS system and selected missense mutations thereof.

28. A nucleic acid array consisting essentially of probes from complexes IV and V of the OXPHOS system.

29. The nucleic acid array of claim 28 consisting essentially of probes comprising the following mutations C5911T, G5913A, A5935G, G5973A, G6081A, G6150A, T6124C, T6253C, G6261A, G6267A, G6285A, C6340T, G6480A, A6663G, G7041A, T7080C, A7083G, A7158G, A7305C, and T7389C.

30. A method for identifying a subject likely to have or at risk of developing cancer, said method comprising determining the mitochondrial haplogroup of said subject and if the subject's mitochondrial haplogroup is U, identifying said subject as likely to have or at risk of developing cancer.

31. The method of claim 30 wherein said cancer is prostate or kidney (renal) cancer.

32. A method for identifying a subject likely to have or at risk of developing cancer, said method comprising determining the mitochondrial haplogroup of said subject to be L0, and identifying the presence of the missense COI mutation T7389C.
